(19) United States
(12) Patent Application Publication
(10) Pub. No.: US 2022/0047538 A1
(43) Pub. Date: Feb. 17, 2022

(54) TUFT CELL SPECIFICATION IN INFLAMMATORY ILEITIS

(71) Applicant: VANDERBILT UNIVERSITY, Nashville, TN (US)

(72) Inventors: Amrita BANERJEE, Nashville, TN (US); Ken S. LAU, Nashville, TN (US)

(21) Appl. No.: 17/428,734

(22) PCT Filed: Feb. 6, 2020
(86) PCT No.: PCT/US2020/016991

§ 371 Date: Aug. 5, 2021

Related U.S. Application Data
(60) Provisional application No. 62/802,044, filed on Feb. 6, 2019.

(51) Int. Cl. A61K 31/194; A61P 1/00; A61P 29/00; A61K 45/06; A23L 33/10

(52) U.S. Cl. CPC A61K 31/194; A61P 1/00; A23V 2002/00; A61K 45/06; A23L 33/10; A61P 29/00

(57) ABSTRACT

Methods for preventing, treating or suppressing an inflammatory bowel disease, particularly Crohn's Disease are disclosed. The methods include administering a pharmaceutical composition that enhances tuft cell specification. The pharmaceutical composition can comprise succinic acid or a pharmaceutically acceptable salt, ester, solvate, or prodrug thereof.

### STATEMENT ACKNOWLEDGING OF GOVERNMENT SUPPORT

This invention was made with government support under grant no. R01DK103831 awarded by the National Institutes of Health. The Government has certain rights in the invention.

### BACKGROUND

Inflammatory Bowel Disease (IBD) and its subtype Crohn's disease (CD) arise due to a loss of tolerance to environmental antigens in genetically susceptible individuals. Longitudinal analysis of CD incidence has identified an inverse correlation between rates of communicable disease and autoimmune disorders, particularly in countries endemic for helminth infestation. A case report published by Broadhurst et al described the use of helminth eggs to treat an IBD patient with refractory disease. Induction of a type 2 immune response following helminth colonization promoted mucosal healing and achieved clinical remission. Epithelial tuft cells are responsible for orchestrating the type 2 immune response following helminth colonization via the release of the type 2 cytokine IL-25. In acute infection, tuft cells drive their own specification and tuft cell hyperplasia is a host response for worm extrusion. Therefore, modulation of tuft cell function may prove efficacious in CD treatment.

Crohn's disease (CD) is a relapsing-remitting Inflammatory Bowel Disease (IBD) characterized by chronic inflammation of the small intestine, with 60% of CD patients developing disease in the terminal ileum. Despite the rising rate of IBD diagnoses, the etiology of CD remains unclear but is thought to be partially driven by a combination of a compromised barrier, a dysbiotic microbiome, and an altered immune response. Genome-wide association studies have clearly implicated epithelial-specific genes regulating microbial tolerance and clearance in IBD pathogenesis. In light of this, therapeutic strategies, including antibiotics and probiotics, to manipulate the microbiome for ameliorating disease activity have been attempted with limited success. Long-term remission is often not achieved with antibiotic therapy as patients often develop intolerance and other side effects to prolonged antibiotic regimens. Similarly, neither the VSL3 probiotic, which consists of eight lactic acid-producing bacterial species, nor fecal microbiome transplants have demonstrated the ability to induce response and remission in the majority of IBD patients, though no significant adverse effects have been reported from either treatment strategy. Since non-targeted microbiome therapies have had limited efficacy in achieving remission in CD, the development of targeted therapies may benefit CD treatment.

Longitudinal analysis of global IBD incidence has identified an inverse correlation between the rates of communicable disease and autoimmune disorders. Known as the “hygiene hypothesis,” this phenomenon is thought to result from improved hygiene practices associated with decreased tolerance to environmental antigens. This paradoxical effect has led to emerging interest in the use of helminths, or parasitic worms, for the treatment of IBD. Studies of the gut mucosa in major subsets of CD patients have demonstrated an increase in proinflammatory cytokines related to T-helper (Th)1 and Th17 cells, including interferon-γ and interleukin (IL)-17 as well as a commensurate decrease in Th2-associated cytokines, such as IL-25 and IL-13. An enhanced type 2 immune response, such as that seen in helminth infection, has been shown to suppress Th1 and Th17 activity. Clinical trial data from CD and ulcerative colitis (UC) patients have been inconclusive, as some trials have demonstrated decreased disease activity while others have been discontinued due to lack of efficacy. Moreover, helminth therapy has its drawbacks given prolonged infection can cause complications.

There is an ongoing unmet need for in the art for compositions and methods for treating IBD and its subtype CD. The present disclosure addresses these and other needs.

### SUMMARY

Disclosed herein are compounds, compositions, and methods for treating or suppressing an inflammatory bowel disease (IBD). In some cases, the IBD is Crohn's disease such as Crohn's ileitis. In some cases, the compounds and compositions disclosed herein can enhance tuft cell hyperplasia independent of helminth colonization. In some cases, the compounds and compositions disclosed herein can induce metabolite changes that are responsible for driving tuft cell hyperplasia independent of helminth colonization.

The compounds and compositions disclosed herein for treating or suppressing IBD can include succinic acid or a pharmaceutically acceptable derivative, prodrug, ester, salt, or solvate thereof. Pharmaceutical compositions comprising a therapeutically effective amount of succinate or a pharmaceutically acceptable derivative, prodrug, ester, salt, or solvate thereof for treating IBD are also disclosed.

As disclosed herein, the compounds and compositions can treat IBD by increasing tuft cell number. Accordingly, disclosed herein are methods of suppressing IBD and increase tuft cell number in a subject by administering succinate or a pharmaceutically acceptable derivative, prodrug, ester, salt, or solvate thereof to the subject.

Administering the pharmaceutical composition can incude providing the composition in drinking water consumed by the subject. For example, succinate or the pharmaceutically acceptable derivative, prodrug, ester, salt, or solvate thereof can be provided at a concentration of 100 mM or greater such as from 10 mM to 500 mM, or from 100 mM to 200 mM in the subject's drinking water.

### DESCRIPTION OF THE FIGURES

FIGS. 1A-1I show human tuft cell number is decreased in biopsies of inflamed tissue from patients with ileal Crohn's disease. (FIG. 1A) Immunofluorescence staining of pEGFR(Y1068) and COX2 in human ileal tissues from healthy and Crohn's disease patients. Co-localization of pEGFR and COX2 is used to identify small intestinal tuft cells, demarcated by white arrows. Magnified inset of the epithelium shows that both markers are expressed in individual tuft cells, demarcated by gray arrows. Representative images are shown from two separate normal or Crohn's disease patients, respectively. Hoechst denotes nuclei, scale bar=100 μm. (FIG. 1B) Quantification of pEGFR and COX2 double-positive tuft cells in normal and Crohn's disease ileal samples. Each dot represents a separate sample. Error bars represent SEM for n=11 normal and n=14 Crohn's disease samples, respectively. *p-value<0.05 by t-test. (FIG. 1C) Histology of distal ileum from wildtype, low and high inflammation TNF^{ΔARE/+}, and AtohKO animals. Scale bar=100 μm. (FIG. 1D) Immunofluorescence imaging of MUC2 and LYZ1. Hoechst denotes nuclei, scale bar=100 μm. (FIG. 1E) Immunofluorescence imaging of DCLK1 and Hoechst. Scale bar=100 μm. (FIG. 1F) Immunofluorescence imaging of MPO and Hoechst. Scale bar=100 μm. (FIG. 1G) Quantification of MUC2 staining normalized by Hoechst area in villi. Error bars represent SEM from n=3 mice per condition. **p-value<0.01, *p-value<0.05 by t-test. (FIG. 1H) Quantification of LYZ1 staining normalized by Hoechst area per crypt. Error bars represent SEM from n=4 wildtype and TNF^{ΔARE/+} mice and n=3 AtohKO animals **p-value<0.01, *p-value<0.05. (FIG. 1I) Tuft cell number per villi in the TNF^{ΔARE/+} ileum stratified by MPO+ neutrophils. Low MPO<35 neutrophils and High MPO 35 neutrophils. Error bars represent SEM for n=40 villi per condition across 6 TNF^{ΔARE/+} animals **p-value<0.01 by t-test.

FIGS. 2A-2I show p-creode trajectory analysis of ileal epithelial scRNA-seq data supports an alternate origin for small intestinal tuft cells. (FIGS. 2A-C) t-SNE analysis of scRNA-seq data generated from (FIG. 2A) wildtype, (FIG. 2B) TNF^{ΔARE/+}, and (FIG. 2C) AtohKO ileal epithelium. Cell type clusters, including goblet cells, Paneth cells, enteroendocrine cells, tuft cells, enterocytes, and stem/progenitor cells, were identified by kmeans clustering and manually annotated. Each t-SNE plot depicts 1,450 randomly selected datapoints from their corresponding complete dataset and each datapoint represents a single cell. (FIG. 2D) Quantification of tuft cell percentage within the scRNA-seq datasets of wildtype, TNF^{ΔARE/+}, and AtohKO ileal epithelium. Error bars are generated from n=6 wildtype replicates, n=3 TNF^{ΔARE/+} replicates, and n=3 AtohKO replicates. **p-value<0.01, *p-value<0.05, and ns (not significant) by t-test. (FIGS. 2E-G) p-Creode analysis of scRNA-seq datasets shown in FIGS. 2A-C, depicting the most representative topology map over n=100 runs for (FIG. 2E) wildtype, (FIG. 2F) TNF^{ΔARE/+}, and (FIG. 2G) AtohKO datasets. Graph overlay depicts ArcSinh-scaled Dclk1 gene expression data. Cell lineages, including secretory cells, absorptive cells, tuft cells, and stem cells, were manually labelled. Node size represents cell state density and each edge represents cell state transitions. (FIG. 2H) Quantification of n=100 p-Creode maps for wildtype, TNF^{ΔARE/+}, and AtohKO datasets, respectively. Tuft cell placement was classified as secretory (grey) when the tuft cell lineage shared a trajectory with the secretory lineage and as non-secretory when the tuft cells and absorptive lineage shared a trajectory. (FIG. 2I) Heatmap depicting z-score normalized expression of tuft cell gene signature in tuft and non-tuft cell populations in both wildtype and AtohKO datasets. All values are statistically significant, ***p-value<0.001 by t-test.

FIGS. 3A-3S show analysis of AtohKO tuft cell gene expression identified upregulation in metabolic pathways. (FIG. 3A) Heatmap of gene expression trends across pseudotime in tuft cell lineage from wildtype and AtohKO p-Creode topologies. Genes were clustered by their dynamics—Groups 1-2 included upregulated genes and Groups 3-4 included downregulated genes. Group 5 (*) in AtohKO consisted of genes that were 0 expression. (FIG. 3B) KEGG enrichment bar plots for genes that class switch from lower order in wildtype tuft cells to higher order in AtohKO tuft cells. Functional groups were ordered by NES. (FIGS. 3C-H) Trend dynamics along pseudotime of citrate cycle-related genes for the wildtype and AtohKO tuft cell lineages. Solid lines represents wildtype and AtohKO gene expression trends from 10 representative p-Creode graphs. Raw data is shown for wildtype (circles) and AtohKO (diamonds). Confidence interval of raw data was depicted by dashed lines for wildtype and AtohKO. Dynamic time warping was used to fit the wildtype and AtohKO tuft cell data to the same scale. Statistical analysis of trend differences and consensus alignment was performed between conditions, ****p<0.0001 by t-test. (FIG. 31) Gene set enrichment analysis of median difference between wildtype (n=58 cells) and AtohKO (n=64) tuft cell populations. Top 20 gene sets from positive gene enrichment are ranked by the normalized enrichment score (NES) and p-value. Yellow highlighted gene sets are related to the citric acid cycle and metabolism pathways. (FIGS. 3J-K) Positive enrichment plots for the gene sets with the highest NES based on GSEA. (FIGS. 3L-M) Pathway analysis between wildtype and AtohKO tuft cells for (FIG. 3L) gene ontology and (FIG. 3M) KEGG analysis shows positive enrichment for the Tricarboxylic or citrate acid cycle Metabolic Process. (FIGS. 3N-S) Relative expression of TCA cycle genes in wildtype and AtohKO tuft cells. Error bars represent SEM from wildtype (n=58 cells) and AtohKO (n=64 cells). ****p<0.0001, <0.001, **p<0.01, and *p<0.05 by t-test.

FIGS. 4A-4O show in vivo tuft cell hyperplasia in the AtohKO small intestine is microbiome dependent. (FIGS. 4A-B) Representative immunofluorescence staining of (FIG. 4A) LYZ1 and (FIG. 4B) DCLK1 in the AtohKO ileum, (I) without antibiotics and with (II) low dose, (III) mid dose, and (IV) high dose antibiotics. Hoechst denotes nuclei, scale bar=100 μm. (FIGS. 4C-H) Relative expression of TCA cycle genes in AtohKO—no antibiotics, AtohKO—low dose antibiotics, and AtohKO—mid dose antibiotics (dashed) tuft cells. Error bars represent SEM from untreated AtohKO (n=25 cells), low-dose antibiotic-treated AtohKO (n=202 cells), and mid-dose antibiotic-treated AtohKO (n=36 cells). ***p-value<0.001, *pvalue<0.05, and not significant (ns) by t-test. (FIGS. 4I-H) Relative concentration of short chain fatty acids, (FIG. 4I) succinate, (FIG. 4J) malate, (FIG. 4K) butyrate, and (FIG. 4L) fumarate in cecal luminal contents and whole tissue from wildtype and AtohKO animals Error bars represent SEM across n=5 wildtype and n=3 AtohKO replicates. **p-value<0.01 and not significant (ns) by test. (FIG. 4M) Relative succinate concentration in cecal luminal contents among wildtype, untreated AtohKO, and antibiotic-treated AtohKO (magenta, dashed) animals Error bars represent SEM across n=5 wildtype, n=3 AtohKO, and n=3 antibiotic-treated AtohKO replicates. **p-value<0.01 by t-test. (FIG. 4N) PICRUSt functional analysis of 16s profiles from wildtype and AtohKO replicates. Heatmap of z-score normalized relative abundances of statistically significant predicted functional categories from wildtype and AtohKO 16s data. *p-value<0.05 by test. (FIG. 4O) Relative abundance of relevant genus level categories contributing to the “Chlorocyclohexane and chlorobenzene degradation category” between wildtype and AtohKO microbiome profiles. Error bars represent SEM from n=4 wildtype and n=3 AtohKO replicates. **p-value<0.01 and not significant (ns) by t-test.

FIGS. 5A-5J show therapeutic succinate treatment mitigates inflammation in the TNF^{ΔARE/+} model. (FIG. 5A) Histology from control and succinate-treated (120 mM) TNF^{ΔARE/+} ileum. Scale bar=100 μm. (FIGS. 5B-C) Pathological scoring of (FIG. 5B) depth of inflammation (0-4) and (FIG. 5C) tissue injury (0-4) in control and succinate-treated TNF^{ΔARE/+} mice. Error bars represent SEM from n=5 control and n=7 succinate-treated TNF^{ΔARE/+} mice. ***p-value<0.001 by t-test. (FIGS. 5D-E) Immunofluorescence imaging of (FIG. 5D) MPO and (FIG. 5E) FOXP3 in the ileum of untreated and succinate treated TNF^{ΔARE/+} ileum. Hoechst denotes nuclei, scale bar=100 μm. (FIGS. 5F-G) Quantification of (FIG. 5F) MPO+ and (FIG. 5G) FOXP3+ cells normalized to nuclei number. Error bars represent SEM from n=4-5 untreated and n=4 succinate-treated mice. **p-value<0.01 and ****p value<0.0001. (FIGS. 5H-I) Immunofluorescence imaging of (FIG. 5H) LYZ1 and (I) DCLK1 in the ileal epithelium of untreated and succinate-treated TNF^{ΔARE/+} ileum. Scale bar=100 μm. White arrows indicate DCLK1+ epithelial tuft cells and Hoechst denotes nuclei. (FIG. 5J) Summary diagram of findings.

### DETAILED DESCRIPTION

The compounds, compositions, and methods described herein may be understood more readily by reference to the following detailed description of specific aspects of the disclosed subject matter and the Examples included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “treat” or other forms of the word, such as “treated” or “treatment,” is meant to administer a composition or to perform a method in order to reduce, inhibit, or eliminate a particular characteristic or event. The term “control” is used synonymously with the term “treat.”

The term “therapeutically effective” means the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Examples of prodrugs that can be used to improve bioavailability include esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, sulfenyl, optionally substituted sulfenyl, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally susbstituted hemiaminal, acetal, optionally substituted acetal, hemiacetal, optionally susbstituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, bis-(acyloxybenzyl)esters, optionally substituted bis-(acyloxybenzyl)esters, (acyloxybenzyl)esters, optionally substituted (acyloxybenzyl)esters, and BAB-esters.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from an alkali agent such as sodium, potassium, calcium, magnesium, lithium, or a combination thereof. Other salts include those derived from organic compounds such as arginine, lysine, histidine, ornithine, creatine, agmatine, citrulline, or any combination thereof. Other salts can be derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

“Pharmaceutically acceptable derivative” or “pharmaceutically acceptable salt” refers to a prodrug or salt that is pharmaceutically acceptable and has the desired pharmacological properties. Such derivatives or salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

Effective amounts of a compound or composition described herein for treating a mammalian subject can include about 0.1 to about 25,000 mg/Kg of body weight of the subject/day, such as from about 1 to about 1000 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, therapeutic agents, compositions, and methods, examples of which are illustrated in the accompanying Examples.

Compounds and Compositions

Provided herein are compounds, compositions, and methods for treating an inflammatory bowel disease. The compounds disclosed herein can be agents that enhance the activity and/or expression of a tuft cell. For example, the compounds can be agents that enhance tuft cell hyperplasia or tuft cell specification. In some embodiments, the compounds can be agents that trigger release of IL-25, IL-22, and/or IL-13 by tuft cells.

In some examples, the compounds can be an exogenously derived metabolite or compound of the tricarboxylic acid cycle. In specific examples, the compound can include succinic acid, salts, prodrugs, esters, analogs or derivatives thereof, or any combination thereof. The term “succinic acid” as used herein refers to the dicarboxylic acid HOOCCH₂CH₂COOH formed in the Krebs cycle and various fermentation processes. The term “succinic acid” as used herein is synonymous with the term “succinate.” Chemically, succinate corresponds to a salt or a derivative such as an ester derivative of succinic acid. Thus, succinate and succinic acid refer to the same compound that can exist in either of two forms.

In some examples, the compounds can be a salt or prodrug of succinic acid, preferably a water-soluble salt or prodrug thereof. As described herein, the salt can be an alkali based succinate salt such as sodium succinate or potassium sodium succinate. In some embodiments, the compound can include sodium succinate hexahydrate. The prodrug can be a derivative of the active compound which is converted after administration back to the active compound. More particularly, it can be a derivative of the compounds disclosed herein which may be active drugs and/or which are capable of undergoing hydrolysis (of for example, an ester or methyleneoxy ester moiety) or cleavage so as to release active free drug. The physiologically hydrolyzable groups serve as prodrugs by being hydrolyzed in the body to yield the parent drug per se.

As described herein, the compounds and compositions can be exogenously derived. The term “exogenous,” as used herein, refers to a substance or molecule originating or produced outside of a subject such as an organism. An exogenous compound can be synthesized or may be from the same or a different species.

Pharmaceutical Compositions

The compounds described herein or pharmaceutically acceptable salt or solvate thereof, a derivative thereof, or a prodrug thereof can be provided in a pharmaceutical composition. In some embodiments, the composition can include a pharmaceutically acceptable salt of succinic acid.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of a compound described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include water, saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

Liquid dosage forms for oral administration of the compounds described herein or derivatives thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents. Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

In some examples, the active compounds can be added to a beverage such as drinking water, flavored water, a shot beverage, a multi-dose beverage, tea, concentrated fruit juice, straight juice, carbonated drink, soft drink, or milk beverage.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner Examples of embedding compositions that can be used are polymeric substances and waxes. The active compounds can also be in micro-encapsulated form, if appropriate, with one or more of the above-mentioned excipients. The disclosed compounds can also be incorporated into polymers, examples of which include poly (D-L lactide-co-glycolide) polymer for intracranial tumors; poly[bis(pcarboxyphenoxy) propane:sebacic acid] in a 20:80 molar ratio (as used in GLIADEL); chondroitin; chitin; and chitosan.

In certain embodiments, it is contemplated that the oral compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release delivery systems. The active compound can be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid compound dispersed in a solvent-based polymer solution (suspension method), or by dissolving the compound in a solvent-based polymer solution (dissolution method). One can attach poly(ethylene glycol) to proteins (PEGylation) to increase the in vivo half-life of circulating compound.

Compositions containing an active compound described herein or derivatives thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught herein.)

The effective amount of the compounds and compositions or pharmaceutically acceptable salts, derivatives or prodrugs thereof as described herein can be determined by one of ordinary skill in the art and includes exemplary dosage amounts for a mammal of from about 0.5 to about 300 mg/kg of body weight of active compound per day. Alternatively, the dosage amount can be from about 0.5 to about 250 mg/kg of body weight of active compound per day, from about 0.5 to about 200 mg/kg of body weight of active compound per day, from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 0.5 mg/kg of body weight of active compound per day, or about 1 mg/kg of body weight of active compound per day. The expression effective amount, when used to describe an amount of compound in a method, refers to the amount of a compound that achieves the desired pharmacological effect or other effect, for example an amount that results in reduction of a behavioral correlate to inflammatory bowel disorder, an amount that results in reduction of a correlate to Crohn's disease, or treatment of inflammatory bowel disease.

In some examples, the pharmaceutical composition can be formulated for oral delivery. For example, the pharmaceutical composition can be delivered in a food or drink product. When formulated as a liquid, the active ingredient (succinate or a pharmaceutically acceptable salt, ester, solvate, or prodrug thereof) can be provided at a concentration of 50 mM or greater, e.g., 75 mM or greater, 100 mM or greater, 110 mM or greater, 120 mM or greater, 130 mM or greater, 150 mM or greater, 160 mM or greater, from 50 mM to 200 mM, from 100 mM to 200 mM, or from 100 mM to 150 mM.

Those of skill in the art will understand that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Administration of the compounds and compositions described herein or pharmaceutically acceptable salts thereof to a subject can be carried out using therapeutically effective amounts of the compounds and compositions described herein or pharmaceutically acceptable salts thereof as described herein for periods of time effective to treat a disorder.

Kits

Also provided herein are kits for treating inflammatory bowel disease in a subject. A kit can include any of the compounds or compositions described herein. A kit can further include one or more conventional drug for treating inflammatory bowel disease. A kit can include an oral formulation comprising any one or more of the compounds or compositions described herein. A kit can additionally include directions for use of the kit (e.g., instructions for treating a subject with inflammatory bowel disease).

Methods of Use

Intestinal tuft cells possess the capacity to modulate intestinal inflammation. Decreased numbers of tuft cells in ileal tissues were observed in patients with inflammatory bowel disease, particularly Crohn's disease. Tuft cell presence has been shown to suppress inflammation and increasing tuft cell specification counteracts pro-inflammatory signals in the inflamed intestine.

Provided herein are methods of preventing, treating, or suppressing inflammatory bowel disease in a subject. These methods can include administering a tricarboxylic acid intermediate or metabolite to the subject at risk for, or having, an inflammatory bowel disease. In particular, the methods can include administering to a subject an effective amount of one or more of the compounds or compositions described herein, or a pharmaceutically acceptable salt or prodrug thereof. The compounds and compositions described herein or pharmaceutically acceptable salts thereof are useful for treating inflammatory bowel disease in mammals such as in humans. In some embodiments, the compositions disclosed herein can enhance tuft cell specification. The term “cell specification” as used herein, refers to the process a cell undergoes in which the cell gives rise to a progenitor cell committed to forming a particular limited range of differentiated cell types. Committed progenitor cells are often capable of self-renewal or cell division. In some embodiments, the compositions disclosed herein can enhance tuft cell hyperplasia. The term “hyperplasia” as used herein is intended to refer to an abnormal or unusual increase in growth or division of tuft cells composing a tissue or organ, particularly in the intestine. Tuft cell hyperplasia and specification may stimulate pathway in intestinal cells to release IL-27, IL-25, IL-22, and/or IL-13 and enhance an immune response in the subject. In one embodiment, the subject is treated with succinic acid or a pharmaceutically acceptable ester, salt, or prodrug thereof, as described herein. In other embodiments, the compound or composition is a succinate analog or another agent that enhance tuff cell specification.

As described herein, the subject can be treated by the methods disclosed herein an inflammatory bowel disease such as Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some examples, the subject is being treated for Crohn's disease, e.g., Crohn's ileitis.

As described herein, the compounds and compositions can be adapted for oral, rectal, topical, vaginal, parenteral, intramuscular, intraperitoneal, intraarterial, intrathecal, intrabronchial, subcutaneous, intradermal, intravenous, nasal, buccal or sublingual routes of administration. For oral administration, particular use is made of liquid formulations, such as in the subject's standard drinking water.

The amount of the active composition or compound used in each method and present in each effective dose is selected with regard to consideration to the half-life of the compound, the identity and/or stage of the inflammatory disease, the patient's age, weight, sex, general physical condition and the like. The amount of active component required to enhance tuft cell specification without significant adverse side effects varies depending upon the pharmaceutical composition employed and the optional presence of other components. Generally, a useful therapeutic dosage is administered at a concentration of from and including dosages of from 1 mg/day to 120 g/day, from 1 mg/day to 50 g/day, from 100 mg/day to 25 g/day, or from 1 g/day to 20 g/day, active compound for adults. In one embodiment, the selected composition is administered in a single dose. In another embodiment an initial dose of a composition may be optionally followed by repeated administration for a duration selected by the attending physician.

Dosage frequency may also depend upon the factors identified above, and may range from 1 to 6 doses per day for a duration of at least 3 days, at least 5 days, at least 10 days, at least 15 days, at least 20 days, at least 25 days, at least 30 days, at least 40 days, at least 50 days, or at least 60 days. In some embodiments, the pharmaceutical composition can be administered daily for at least 5 days up to 90, at least 10 days up to 90, or at least 10 days up to 30. The compounds may also be incorporated into other infection control protocols, involving repetitive cycles of dosing. Selection of the appropriate dosing method would be made by the attending physician.

In further examples, the compounds and compositions disclosed herein can be used in combination with one or more other pharmaceutically active agents. In such cases, the compounds and compositions can be administered consecutively, simultaneously or sequentially with the one or more other pharmaceutically active agents. In some examples, the additional therapeutic agent can include an antibiotic.

### EXAMPLES

### Commensal-Derived Succinate Enhances Tuft Cell Specification and Suppresses Ileal Inflammation

Longitudinal analysis of Crohn's disease (CD) incidence has identified an inverse correlation with helminth infestation and it has been shown that intestinal tuft cell hyperplasia is needed for helminth response. Tuft cell frequency was decreased in the inflamed ilea of CD patients and a mouse model of TNFα-induced Crohn's-like ileitis (TNF^{ΔARE}). Single-cell RNA sequencing paired with unbiased differential trajectory analysis of the tuft cell lineage in a genetic model of tuft cell hyperplasia (AtohKO) demonstrated that the tuft cell lineage had increased tricarboxylic acid (TCA) cycle gene signatures. Commensal microbiome-derived succinate was detected in the ileal lumen of these animals while microbiome depletion suppressed tuft cell hyperplasia. Therapeutic succinate treatment in TNF^{ΔARE} animals reduced pathology in correlation with induced tuft cell specification. Provide herein is evidence implicating the modulatory role of intestinal tuft cells in chronic intestinal inflammation, which could facilitate leveraging this rare and elusive cell type for CD treatment. In particular, it was demonstrated that tuft cell specification is decreased in the inflamed small intestine of both human and mouse. By analyzing molecular pathway alterations in a lineage-specific manner during tuft cell hyperplasia, commensal microbial-driven changes in metabolic pathways that promote tuft cell specification were identified. Finally, metabolite-induced tuft cell hyperplasia in a mouse model of Crohn's-like ileitis suppressed disease symptoms and restored epithelial architecture.

Methods

Human Tissue: Formalin-fixed, paraffin-embedded blocks of ileum surgical resections were obtained from the Vanderbilt Cooperative Human Tissue Network Western Division, along with deidentified patient data and pathology reports. All studies were performed according to Vanderbilt University Institutional Review Board No. 182138. Pathological examination was utilized to classify samples as “normal” (n=14) or “diseased” (n=19). Samples from patients with Crohn's disease were included only if inflammation was evident in the distal ileum. Tissue samples were prepared for immunofluorescence imaging as described below.

Mouse Experiments: All animal protocols were approved by the Vanderbilt University Animal Care and Use Committee and in accordance with NIH guidelines. Lrig1^{CreERT2} and Atoh1^{flox/flox} strains, each in a C57BL/6 background, were purchased from Jackson Laboratory to generate Lrig1^{CreERT2/+}; Atoh1^{fl/fl} (AtohKO) animals Cre recombinase activity was induced in two- or three-month-old Lrig1^{CreERT2/+}; Atoh1^{fl/fl} and Lrig1^{+/+}; Atoh1^{fl/fl} males via intraperitoneal administration of 2 mg of tamoxifen (Sigma-Aldrich) for four consecutive days. Mice were sacrificed and tissues were harvested twenty-one days after the first injection. TNF^{ΔARE/+} and wildtype littermates were sacrificed at four months of age after disease onset, followed by tissue collection Animal weights were recorded at regular intervals.

For microbiome depletion experiments, Lrig1^{CreERT2/+}; Atoh1^{fl/fl} animals were pre-treated with a broad-spectrum antibiotic cocktail containing kanamycin (4.0 mg/ml), metronidazole (2.15 mg/ml), gentamicin (0.35 mg/ml), colistin sulfate (8500 U/ml), and vancomycin (0.45 mg/ml) or ampicillin (1 mg/ml) in their drinking water for seven days prior to tamoxifen treatment. Mid-dose antibiotics and low-dose antibiotics were 0.75× and 0.25× of the original 1× concentration, respectively. Following tamoxifen administration, Lrig1^{CreERT2/+}; Atoh1^{fl/fl} received either standard or antibiotic-supplemented drinking water for an additional fourteen days. For microbiome experiments, co-housed male Lrig1^{CreERT2/+}; Atoh1^{fl/fl} littermates received either vehicle (corn oil) or tamoxifen for 1 month. For succinate treatment, TNF^{ΔARE/+} received either sodium succinate hexahydrate (120 mM; Alfa Aesar) or standard drinking water following disease onset (three- to four-months-old) for five days or one month.

Immunofluorescence staining and imaging: Paraffin-embedded ileal tissues were section (5 μm) prior to deparaffinization, rehydration, and antigen retrieval using a citrate buffer (pH 6.0) for 20 minutes in a pressure cooker at 105° C., followed by a 20-minute cool down at room temperature (RT). Endogenous background signal was quenched by incubating tissue slides in 3% hydrogen peroxide for 10 minutes at RT. Tissue sections were blocked in staining buffer (3% bovine serum albumin/10% normal donkey serum) for 1 hour at RT prior to incubation with primary antibody overnight at RT. Antibodies used for immunofluorescence included those against LYZ1 (DAKO, 1:100, rabbit), MUC2 (Santa Cruz, 1:100, rabbit), MPO (DAKO, 1:100, rabbit), DCLK1 (Santa Cruz, 1:100, goat), FOXP3 (eBioscience, 1:50, rat), and GATA3 (Abcam, 1:50, rabbit). Sections were then incubated with AlexaFluor (AF)-555- or AF-647-conjugated secondary antibodies (Life Technologies, 1:500) for 1 hour at RT and Hoechst (1:10,000, Life Technologies) for 10 min at RT. For human tuft cell labeling, AF-488-conjugated pEGFR (Abcam, 1:100) and unconjugated COX2 (CST, 1:100, rabbit) were used. Slides were incubated with anti-rabbit AF-647-conjugated secondary antibody and stained with Hoechst. Imaging was performed using a Zeiss Axio Imager M2 microscope with Axiovision digital imaging system (Zeiss, Jena GmBH, Germany).

Image quantification: To quantify human tuft cells, the number of crypt and villus structures were counted per field of view (FOV) for each subject (approximately 15-20 FOVs per sample). Tuft cell number, as identified by pEGFR and COX co-labeling, was manually counted per FOV in a blinded fashion. Quantification of tuft cell number per epithelial structure (crypt and villus) was generated for each subject and then stratified by disease state (“normal” or “Crohn's disease”) based on the pathology report. Results were analyzed by t-test using Prism GraphPad. For MUC2 quantification, manual demarcation of the epithelial villi was performed in each FOV and a nuclear mask was generated based on Hoechst staining. In the same region, a MUC2 mask was generated using immunofluorescence staining of MUC2. Total area of both masks was calculated to generate a normalized ratio of MUC2 intensity to nuclear staining. This process was repeated for LYZ1 quantification, except that only crypts were manually demarcated in the FOVs. For MPO and DCLK1 quantification, each villus was considered a separate unit and number of MPO+ neutrophils and DCLK1+ tuft cells were counted in a blinded fashion. Tuft cell number per villi was stratified based on MPO staining as either “low inflammation” (<35 neutrophils per villi) or “high inflammation” (≥35 neutrophils per villi). Finally, for FOXP3 and MPO quantification, total area of each mask was used to calculate a normalized ratio of signal to nuclear staining.

Immunohistochemistry and histological scoring: Paraffin-embedded ileal tissue from wildtype and succinate-treated slides were retrieved as described above and standard H&E staining was performed for histology. To identify eosinophils, tissues were incubated with anti-MBP (Mayo Clinic Arizona) followed by anti-rat HRP and counterstained with hematoxylin Immunohistochemistry was performed by the Vanderbilt Translational Pathology Shared Resource and 20× brightfield scanning of immunohistochemistry slides was performed by a Leica SCN400 Slide Scanner in the Vanderbilt Digital Histology Shared Resource. Degree of inflammation in the distal ileum was assessed in a blinded fashion by a trained pathologist. Damage to the epithelium was assessed using depth of inflammation on a five-point scale (0=no damage, 1=mucosal damage only, 2=submucosal infiltration, 3=infiltration into muscularis propria, and 4=infiltration into the periintestinal fat), while extent of inflammatory injury in the tissue swiss roll was also scored on a five-point scale (0=<10%, 1=10-25%, 2=25-50%, 3=50-75%, and 4=>75%).

Enteroid Experiment: Ileal tissue was dissected and incubated in chelation buffer (3 mM EDTA/EGTA, 0.5 mM DTT, 1% P/S) at 4° C. for 45 minutes. The tissue was shaken in PBS and filtered through a 100 μm filter to isolate individual ileal crypts. The crypt suspension was centrifuged at 2.8×1000 RPM for 1½ minutes at 4° C. following which 10 μl of crypt pellet was resuspended in 300 μl of reduced growth factor Matrigel and embedded in a 24-well dish. Enteroids were cultured initially in IntestiCult Organoid Growth Medium (StemCell Technologies) supplemented with Primocin antimicrobial reagent (InvivoGen, 1:1000) for 4 days before being changed to Primocin-supplemented differentiation media. For ex vivo Cre-activation, Lrig1^{CreERT2/+} Atoh1^{fl/fl} enteroids were treated overnight at 37° C. with either 1 μM 4-hydroxytamoxifen (Sigma-Aldrich) or vehicle (ethanol) in differentiation media. Enteroids were passaged the following day into 8-well chamber slides and, after 5 days, were fixed on ice using 4% PFA for immunofluorescence staining.

Enteroid Immunofluorescence Staining: Fixed enteroids were permeabilized with Triton X-100 for 30 min and blocked with 1% normal donkey serum (PBS) for 30 min at RT. Enteroids were stained with primary antibodies against LYZ1 (DAKO, 1:100, rabbit), MUC2 (Santa Cruz, 1:100, rabbit), and DCLK1 (Santa Cruz, 1:100, goat). Enteroids were then incubated with AF-555- or AF-647-conjugated secondary antibodies (Life Technologies, 1:500) for and Hoechst (1:10,000, Life Technologies) 1 hour at RT. Vectashield Antifade Mounting Medium (Vectorlabs) was applied to enteroids before imaging with a Nikon Spinning Disk Confocal microscope.

Droplet-based single-cell RNA-sequencing: Ileal crypts from human and mouse tissue were isolated as described above. Crypts were dissociated into single cells using a cold-activated protease (1 mg/ml)/DNAseI (2.5 mg/ml) enzymatic cocktail in a modified protocol that maintains high cell viability. Dissociation was performed at 4° C. for 15 minutes followed by trituration to mechanically disaggregate cell clusters. Cell viability was assessed by counting Trypan Blue positive cells. The cell suspension was enriched for live cells with a MACS dead cell removal kit (Miltenyi) prior to encapsulation. Single cells were encapsulated and barcoded using the inDrop platform (1CellBio) with an in vitro transcription library preparation protocol (Klein et al, 2015). Briefly, the CEL-Seq work flow entailed (1) reverse transcription (RT), (2) ExoI digestion, (3) SPRI purification (SPRIP), (4) Second strand synthesis, (5) T7 in vitro transcription linear Amplification, (7) SPRIP, (8) RNA Fragmentation, (9) SPRIP, (10) primer ligation, (11) RT, and (12) library enrichment PCR. Each sample was estimated to contain approximately 2,500 encapsulated cells.

Following library preparation, the samples were sequenced using Nextseq 500 (Illumina) using a 150 bp paired-end sequencing kit in a customized sequencing run. After sequencing, reads were filtered, sorted by their barcode of origin, and aligned to the reference transcriptome using the inDrop pipeline. Mapped reads were quantified into UMI-filtered counts per gene, and barcodes that corresponded to cells were retrieved based on previously established methods. Overall, from approximately 2,500 encapsulated cells, approximately 1,800-2,000 cells were retrieved per sample.

Pre-processing and batch correction of scRNA-seq data: Datasets were filtered for cells with low library size or high mitochondrial gene expression. Filtered datasets for each replicate were analyzed using the Seurat pipeline. Briefly, count matrices were log scale normalized followed by feature selection of highly variable genes. Canonical correlation analysis (CCA) was used to align replicates based on biological condition using dynamic time warping. Following subspace alignment, modularity optimization (0.8 resolution) was used to identify cell clusters. The ComBat algorithm was then used to batch correct each gene on a per cluster basis. Visual assessment of alignment between replicates was performed using t-SNE analysis.

p-Creode mapping and trajectory analysis: The wildtype dataset, as well as the GSE92332 ileum dataset, were feature selected using the binned variance method and these features were used for all other conditions. Feature selected datasets were analyzed using the p-Creode algorithm (https://github.com/KenLauLab/pCreode). For graph scoring, 100 independent runs were generated from each combined dataset. Overlay of ArcSinh normalized expression data was used to identify cell lineages and quantify tuft cell placement as either “secretory” or “non-secretory.”

Trend analysis overview: Trend analysis was performed to identify gene expression changes in the AtohKO tuft cell lineage compared to the wildtype tuft cell lineage. 10 p-Creode resampled runs were used from the wildtype and AtohKO dataset. The top 2,500 genes (ranked by variance) over the tuft cell trajectory were selected from each of the wildtype and AtohKO datasets. The union of these gene sets (3,420 genes) was used for downstream analysis. The dynamic trend of gene expression for each gene over the tuft cell trajectory was obtained by fitting a linear Generalized Additive Model (GAM) with a normal distribution and an identity link function using 10 splines (Servén & Brummitt, 2018). The fitted curves were then normalized between 0 and 1 for comparison between datasets. For each gene trend, classification of its dynamics was performed by calculating its dynamic time warping distances to 12 reference trends. These categories were then broadly combined into 5 classes: (1) upward, (2) upward transitory, (3) downward transitory, (4) downward, and (5) flat. For the coarse grain analysis, 3 trend classes were formed by combining (1) group 1 and 2 genes into “upward,” (2) group 3 and 4 genes into “downward,” and (3) flat. Trend classification was scored by consensus over 10 resampled runs for both the (A) 5-trend and (B) 3-trend analysis. Genes with high consensus are those with a cumulative sum of 16 between the two classifications (for instance, a gene being grouped in the same trend in 8 out of 10 p-Creode replicates in the 5-trend analysis and in 8 out of 10 p-Creode replicates in the 3-trend analysis). This resulted in 2,004 high-consensus genes being used for downstream over-representation analysis. From the list of high consensus genes, genes which switched from (A) wildtype group 4 to AtohKO group 1, 2, or 3, (B) wildtype group 3 to AtohKO group 1 or 2, or (C) wildtype group 2 to AtohKO group 1 were identified. Over-representation analysis, based on KEGG, Reactome pathway, and Wiki pathway datasets, of upregulated genes was performed in WebGestalt. Using 3-trend analysis, genes that switched from wildtype group 2 (downward) to AtohKO group 1 (upward) were identified and performed over-representation analysis.

Visualization and significance testing of trend analysis: Visualization of trend dynamics was performed using Matlab software for enriched genes from the Reactome Pathway “Citric acid cycle (TCA cycle)” gene list. Each gene plot includes raw expression data from each wildtype or AtohKO p-Creode replicate and the trend line as an average of raw expression data aligned by dynamic time warping across all ten resampled runs for each respective condition. For significance testing between wildtype and AtohKO trends, randomized classifications were generated for each gene in the wildtype and AtohKO condition by resampling from the same distribution for each condition. The null hypothesis stated that there was no consensus across the wildtype classifications or that there was no upward class switching from wildtype to AtohKO trajectories. Simulations comparing the randomized and observed classifications were performed 10,000 times to obtain a p-value.

Gene set enrichment analysis (GSEA) of differential expression: Median difference in gene expression was calculated between wildtype and AtohKO tuft cells. GSEA of differential gene expression was performed to identify positively enriched pathways. Top twenty gene sets with the highest NES and most significant p-value were used for the further analysis. Significance testing by t-test was used to compare relative expression of highly enriched genes between wildtype and AtohKO tuft cells using Prism Graphpad. GSEA and over-representation analysis for specific gene sets (KEGG, Reactome pathway, and Wild pathways) was performed using WebGestalt.

DNA extraction and 16s rRNA sequencing: Ileal luminal contents were collected fresh from tamoxifen- or vehicle-treated Lrig1^{CreERT2/+}; Atoh1^{fl/fl} animals, as described above. All samples were collected on the same day and frozen in 2 ml Eppendorf tubes (DNAse and RNAse free) at −80° C. Microbial genomic DNA extraction was performed using the PowerSoil DNA Isolation Kit (MO BIO Laboratories). Briefly, luminal contents were added to PowerBead Tubes and homogenized twice in a Bead Beater machine for 3 minutes with a 2-minute cool down in between homogenization. Samples were then processed as per the kit instructions and DNA was eluted into a sterile buffer. The V4 region of the 16s rRNA gene from each sample was amplified and sequenced by Georgia Genomics and Bioinformatics Core using the Illumina MiSeq Personal Sequencing platform.

Raw 16S rRNA sequences were filtered for quality (target error rate<0.5%) and length (minimum final length 225 bp) using Trimmomatic (Bolger et al, 2014) and QIIME. Spurious hits to the PhiX control genome were identified using BLASTN and removed. Passing sequences were trimmed of forward and reverse primers, evaluated for chimeras with UCLUST (de novo mode), and screened for mouse-associated contaminant using Bowtie2 followed by a more sensitive BLASTN search against the GreenGenes 16S rRNA database. Chloroplast and mitochondrial contaminants were detected and filtered using the RDP classifier with a confidence threshold of 80%. High-quality 16S rRNA sequences were assigned to a high-resolution taxonomic lineage using Resphera Insight. Functional gene content was inferred using PICRUSt. Statistical analyses between groups was performed using R (v3.5). Heatmap clustering visualizations were generated using Microsoft Excel, selecting microbiome features with FDR adjusted p-values<0.10, and log-transforming values for color scaling.

Pathogen testing: Fresh fecal samples from non-co-housed animals (n=5) and frozen at −20° C. Samples were shipped to IDEXX BioAnalytics (Columbia, Mo.) for pathogen PCR panel and helminth float testing.

O-benzylhydroxylamine derivatization of cecal contents and tissue: O-benzylhydroxylamine (O-BHA) derivatization of common tricarboxylic acid intermediates was performed from both cecal luminal contents and tissues by the Vanderbilt Mass Spectrometry Service Laboratory. Briefly, luminal filtrate was mixed in MeOH/H2O with 0.1% Formic acid and added to Pyr 13C3 (327 μg/ml), EDC, and O-BHA as described in the Sherwood protocol. Tissue homogenates were processed similarly, and both were incubated at room temperature for 1 hour prior to extraction with ethyl acetate. 100 μl of luminal content sample (200 mg/ml) or tissue sample (250 mg/ml) was analyzed for specified metabolites and analyte response ratios were calculated using validated standards. An aliquot of PBS was processed and reconstituted as a negative control.

Results

Reduced tuft cell numbers are correlated to localized inflammation in human and mouse ileum: The role of tuft cells in driving an anti-parasite immune response against helminth infection has been shown. Activation of an anti-parasite response has been implicated in the suppression of the proinflammatory environment in IBD patients. Thus, the correlation between tuft cell numbers and local tissue inflammation in ileal specimens from CD patients was studied. Tuft cell expansion following acute helminth infection has been studied in the small intestine but not the colon and modes of tuft cell specification differ between the two tissues. Therefore, the sample inclusion criteria was restricted to inflamed ileal specimens (n=14) from ileal CD patients compared to normal ileal specimens (n=11) collected from patients without a CD diagnosis, in order to limit analysis to small intestinal tuft cells.

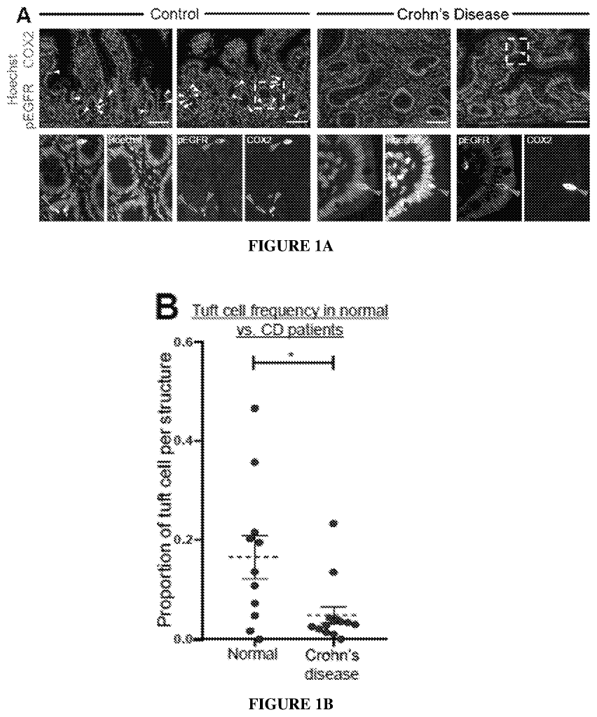

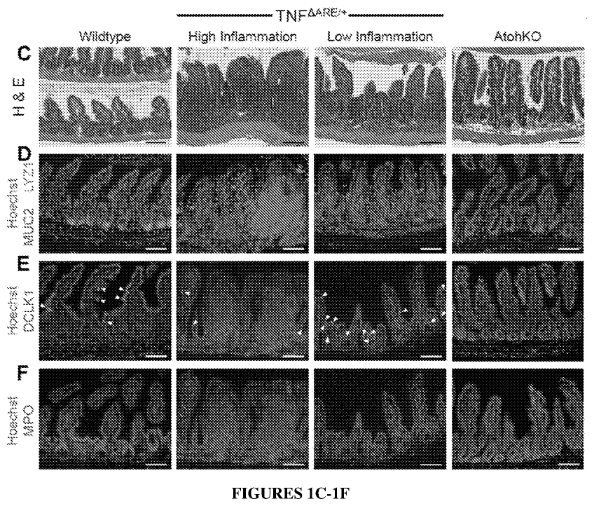

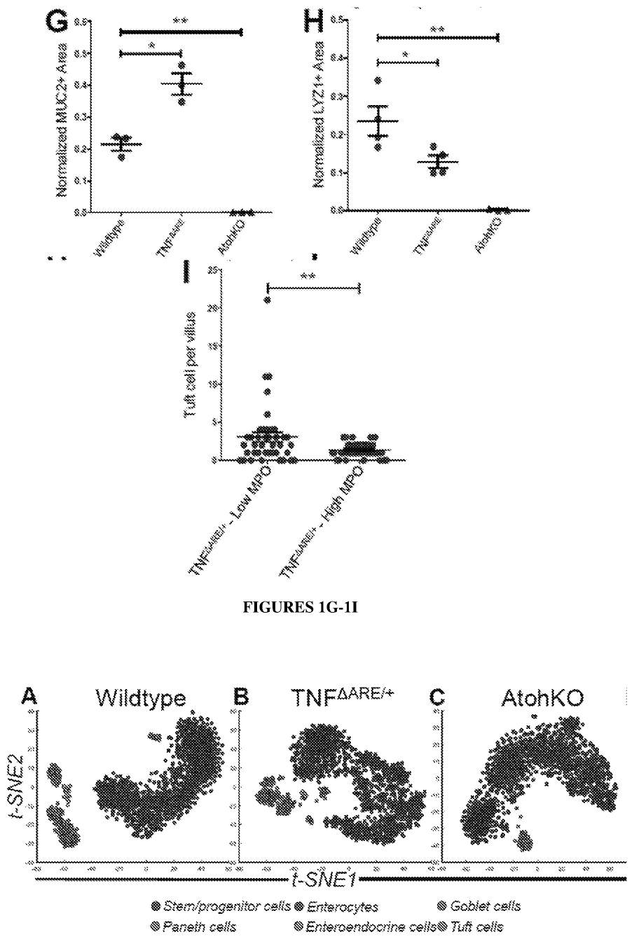

One reason for the absence of studies on human tuft cells is the lack of validated markers. Previous attempts to use antibodies against DCLK1, a tuft cell-specific marker validated in the mouse intestine, to identify human tuft cells have not been successful. Single-cell RNA sequencing (scRNA-seq) data from the normal human ileum showed that the DCLK1 gene was not significantly expressed in human tuft cells, demarcated by TRPM5, compared to murine tuft cells. A double staining strategy of pEGFR(Y1068) and COX2 to specifically mark tuft cells in both mouse and human intestine has been identified. Using this strategy, double-positive pEGFR and COX2 cells in both the villi and crypts of Lieberkühn of the normal ileal epithelium were observed (FIG. 1A), that are distinct from single-positive pEGFR or COX2 cells in the lamina propria. Moreover, many of the double-positive cells possessed a prominent pEGFR-positive apical “tuft,” increasing the likelihood that these were genuine small intestinal tuft cells (FIG. 1A).

This strategy was applied to detect tuft cells from inflamed ileal tissues of CD patients. As expected, inflammation in ileal CD samples was heterogenous and characterized by severe villus blunting. MUC2+ goblet cells were increased in the inflamed epithelium, while LYZ+ Paneth cells were decreased. The few Paneth cells remaining in inflamed regions contained more diffuse lysozyme granules. LYZ expression was increased in the lamina propria in inflamed tissue, most likely from active immune cells. Quantification of tuft cells, detected by co-staining of pEGFR and COX2, revealed a significant reduction in ileal specimens from CD patients (FIGS. 1A-B). Within our sample pool of CD patients, there was no further, progressive decrease in tuft cell number with increased disease severity. In regions with less disease involvement and more organized tissue architecture within CD tissues, tuft cells could still be detected by pEGFR and COX2 staining (FIG. 1A). From these results, it was believed suppression of tuft cell specification may contribute to the loss of inflammation control in CD and thus may be associated with disease development and/or progression.

To assess tuft cell specification in a more controlled manner, the TNF^{ΔARE/+} mouse model was used, which, by the deletion of the AU rich element (ΔARE) in the tumor necrosis factor alpha (Tnfa) gene, has increased levels of TNF-α and develops Crohn's-like ileitis by two to three months of age. The TNF^{ΔARE/+} model mimics many features of human ileal CD, including dependence on TNF-α and microbiome dysbiosis. Histological changes in the terminal ileum of four-month-old TNF^{ΔARE/+} animals was observed, characterized by distorted crypt structure and blunted villi compared to wildtype littermates (FIG. 1C). The number of LYZ1+ Paneth cells was decreased while the remaining ones exhibited diffuse LYZ1 staining, suggesting impaired Paneth cell function (FIGS. 1D, and 1H). The numbers of LYZ1+ cells in the lamina propria and MUC2+ goblet cells in the epithelium were increased (FIGS. 1D and 1G), which bear resemblance to the ilea of human CD patient. Increased immune cell infiltration can be observed by myeloperoxidase (MPO)-positive neutrophils in the lamina propria of inflamed tissues compared to uninflamed controls (FIG. 1F).

Similar to the heterogeneity observed in specimens from CD patients, both highly inflamed and less inflamed regions within the ilea of TNF^{ΔARE/+} mice were observed. Spatially-resolved analysis was performed to determine the relationship between tuft cell numbers and inflammation by quantifying the number of DCLK1+ tuft cells in the epithelium and infiltrating MPO+ neutrophils in the lamina propria on a per-villus basis. Consistent with human intestinal phenotypes, regions classified as highly inflamed with >35 MPO+ cells/villus were characterized by severe villus blunting and distortion, while less inflamed regions (0-35 MPO+ cells/villus) possessed normal crypt-villus architecture and resembled healthy wildtype controls (<10 MPO+ cells/villus). Within the same TNF^{ΔARE/+} animal, tuft cell numbers were significantly decreased in highly inflamed regions compared to less inflamed regions (FIGS. 1E, 1I). The number of tuft cells in less inflamed regions was increased beyond the normal number of tuft cells found in the healthy ileum (FIG. 1E). Therefore, it was concluded that tuft cell frequency is inversely correlated with severity of disease, raising the possibility that increasing tuft cell specification may reduce inflammation.

ATOH1-independent tuft cells are an inducible cell population responsive to the commensal microbiota: Based on our observations in human patients and mouse modeling, it was hypothesized that enhanced tuft cell specification may potentially alleviate inflammation in CD. scRNA-seq has been used to reveal the existence of heterogeneous tuft cell populations, while our group and others have demonstrated an ATOH1-independent origin for small intestinal tuft cell. In the AtohKO model (Atoh1 Knockout—Lrig1^{CreERT2/+}; Atoh1^{fl/fl}) where it was demonstrated that colonic tuft cells are ATOH1-dependent, tuft cells were significantly expanded in a uniform fashion across the small intestine (SF A) in contrast to tuft cell suppression observed in prior studies. Thus, it was hypothesize that the ATOH1-independent tuft cells are a flexible population and can be induced to expand, which presents a viable target that can be manipulated for the treatment of ileal CD. AtohKO intestines possessed normal crypt-villus architecture (SF B) but lacked MUC2+ goblet cells and LYZ1+ Paneth cells (SF C). An increased presence of MPO+ neutrophils in the villi of AtohKO animals (SF D) was observed accompanied by decreased weight gain (SF E), reflecting a baseline level of inflammation potentially due to the loss of microbiome-regulating secretory cell types. These observations led us to question whether ATOH1-independent tuft cell specification is driven by extrinsic cues originating from the luminal microbiota.

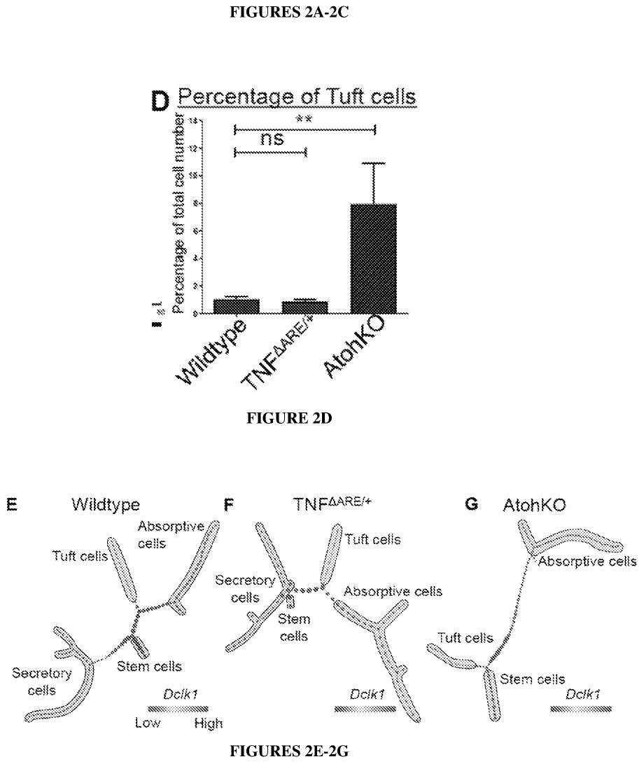

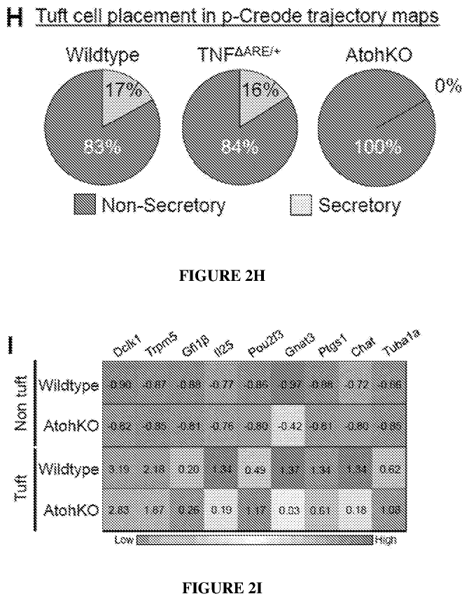

To investigate the nature of luminal perturbation that results in ATOH1-independent tuft cell expansion in the intestine, mouse colony devoid of large parasites known to trigger tuft cell expansion was determined (SF F). To assess the necessity of the commensal microbiota in driving tuft cell expansion, an antibiotic cocktail, consisting of kanamycin, metronidazole, gentamicin, colistin sulfate, and vancomycin, to deplete a broad range of gram-positive and gram-negative bacteria was used. Loss of Atohl in this context, as expected, resulted in the absence of LYZ1+ Paneth cells (FIG. 2A). However, microbiome depletion significantly decreased ATOH1-independent tuft cell expansion in a dose-dependent manner, and at higher doses, completely inhibited ATOH1-independent tuft cell specification (FIG. 2B). However, in wild type mice that possess both ATOH1-dependent and -independent tuft cell populations, microbiome depletion with high dose antibiotics and germ-free housing did not suppress tuft cell specification (FIG. 2C-D). It was reasoned that ATOH1-independent tuft cells are sensitive to luminal changes, while coexisting ATOH1-dependent tuft cells are largely insensitive to these changes.

To verify the heterogeneity of the tuft cell population in a more controlled manner, enteroid systems was used, which are cultured in environments devoid of microbiota. The sterility of the culturing conditions did not inhibit the specification of tuft cells in vehicle-treated control enteroids (FIG. 2), consistent with the independence of tuft cell specification and the microbiota observed when ATOH1 is present in vivo. However, tuft cell specification in sterile enteroids was completely suppressed when Atoh1 loss was induced via Cre recombination (FIG. 2), demonstrating that tuft cell specification observed in the control condition were ATOH1-dependent. Similar to the in vivo condition, loss of Atoh1 also led to suppression of Paneth and goblet cell specification (SF). It was determined that type 2 cytokine interleukin-13 (IL-13) drives tuft cell specification in response to luminal eukaryotic colonization. IL-13 administration in AtohKO enteroids induced tuft cell specification and expansion in an ATOH1-independent manner, at a level similar to IL-13 administration in control enteroids (FIG. 2). These results support the existence of ATOH1-independent and -dependent tuft cell populations, with the former being highly malleable to luminal manipulations.

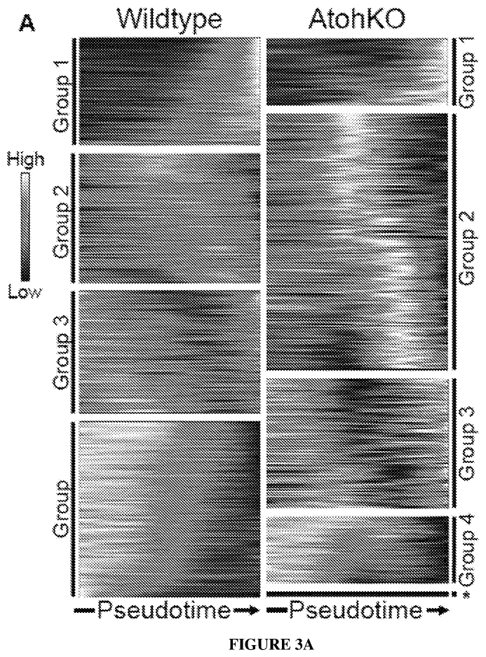

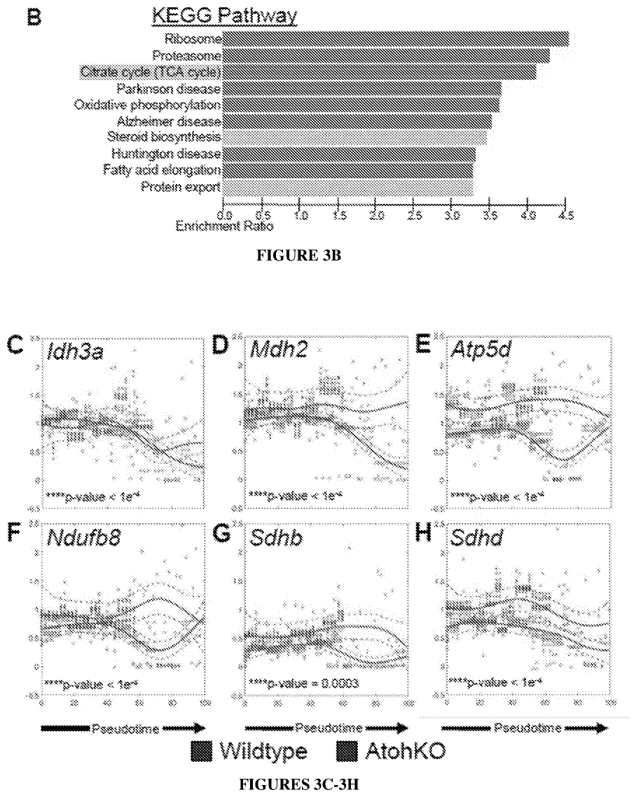

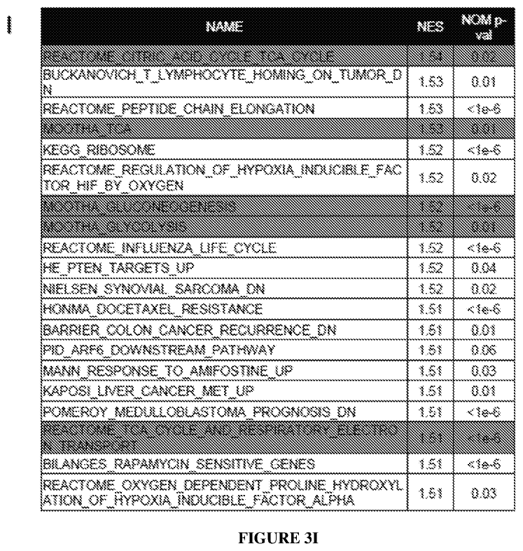

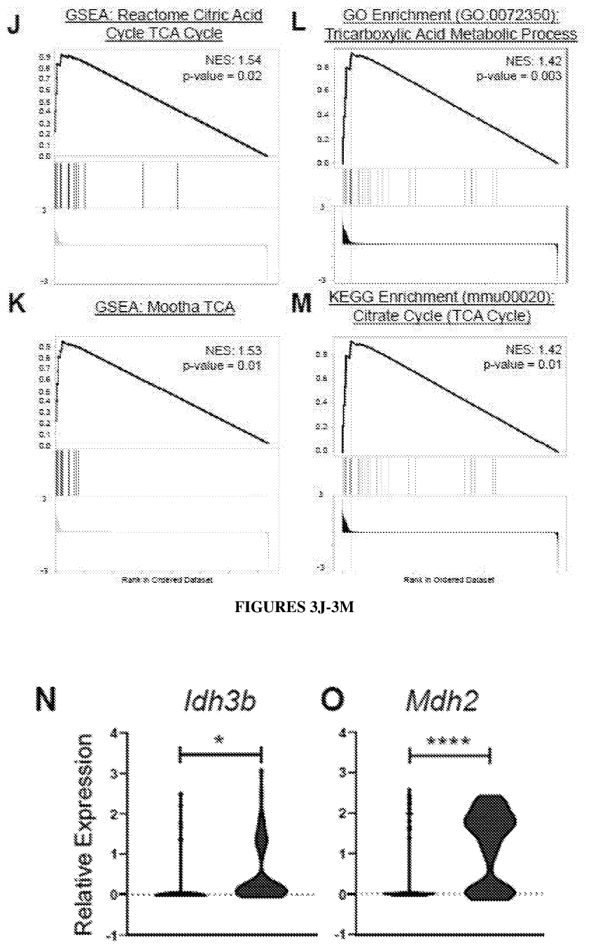

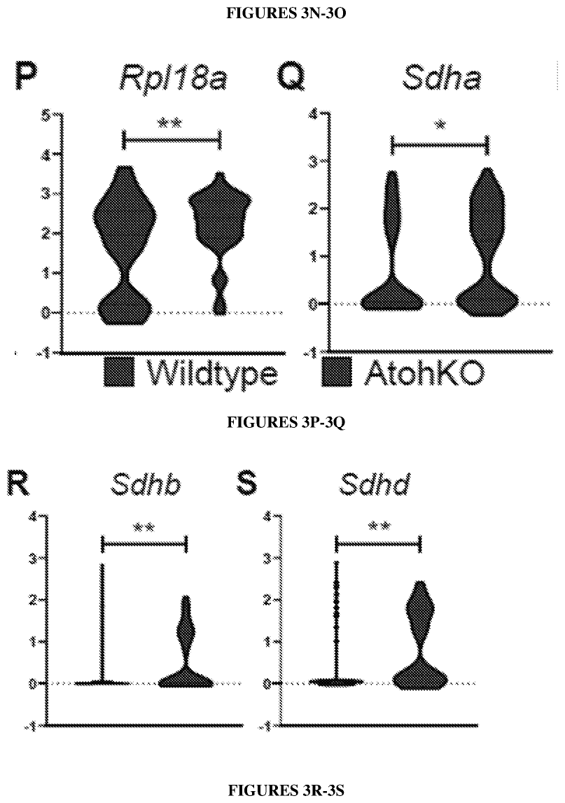

Trajectory analysis of single-cell RNA-sequencing data supports an ATOH1-independent tuft cell specification pathway: To investigate the heterogeneity of tuft cell lineages and associated pathways, scRNA-seq data were generated from the wildtype control, TNF^{ΔARE/+}, antibiotic-treated wild type (ATOH1-dependent only), and AtohKO (ATOH1-independent only) ileal epithelium using the inDrop platform. Following data processing and quality control, 6,932 cells from the wildtype condition (SF), 3,401 cells from the TNF^{ΔARE/+} condition (SF), 3,580 cells from the antibiotic-treated condition (SF), and 2,456 cells from the AtohKO condition (SF), all across multiple biological replicates were obtained. t-SNE and clustering analyses performed on each of the combined datasets demonstrated that all replicates were represented in all cell clusters identified (SF). Stem and progenitor cells, enterocytes, goblet cells, Paneth cells, enteroendocrine cells, and tuft cells were present in the correct proportions in the wildtype condition (FIG. 3). In the TNF^{ΔARE/+} dataset, while all the cell types were represented, the Paneth cell population was greatly diminished, consistent with results observed by immunofluorescence imaging (FIG. 3, SF L). The number of tuft cells was not significantly different between the wildtype and TNF^{ΔARE/+} datasets (FIG. 3), which may be accounted for by the spatial heterogeneity of tuft cells in the inflamed ileum observed by prior microscopy (FIG. 1). Given the loss of spatial resolution subsequent to single-cell dissociation that intersperses inflamed and uninflamed regions, it is reasonable that a global approach such as scRNA-seq cannot accurately capture the spatial heterogeneity of the TNF^{ΔARE/+} model. While other cell populations were relatively stable in the antibiotic-treated wildtype ileum, tuft cell numbers were significantly reduced, consistent with the persistence of a few ATOH1-dependent tuft cells while ATOH1-independent tuft cells were eliminated (FIG. 3, SF). Data from the AtohKO condition confirmed the loss of goblet, Paneth, and enteroendocrine lineages following Atoh1 recombination, consistent with their ATOH1-dependent secretory origins (FIG. 3, SF). Compared to the wildtype tuft cell cluster, the AtohKO tuft cell population was significantly expanded (FIGS. 2C-2D). It was concluded that the scRNA-seq results are largely consistent with epithelial cell populations identified by in situ microscopy analysis.

Tuft cell specification pathways were analyzed using the p-Creode algorithm to produce trajectory representations of scRNA-seq datasets. The wildtype p-Creode map originated from stem cells which bifurcate into the secretory and absorptive lineages (FIG. 2E). As expected, goblet and Paneth cells originated from a common secretory progenitor before diverging into two distinct branches. In contrast, the tuft cell lineage mainly shared a specification trajectory with absorptive cells, rather than the secretory cells (FIG. 2E). In order to evaluate the robustness of this map, 100 p-Creode graphs were generated by randomly sampling the wildtype scRNA-seq dataset and quantified tuft cell placement. p-Creode maps were classified as “secretory” when the tuft cells were grouped with lineages containing goblet cells and as “non-secretory” otherwise. Tuft cell placement was non-secretory in 83% of wildtype trajectories and secretory in the remaining 17% (FIG. 2H). Focused analysis of the tuft cell population in the wildtype ileum revealed two subclasses with divergent gene expression patterns (SF A). Differential expression analysis revealed that subclass A was enriched for metabolism-related genes, including Sdha and Ndufb8, suggesting preferential metabolic activity in this population compared to the second tuft cell subpopulation (SF B). These results have demonstrated the presence of two tuft cell populations, which may explain the varying tuft cell lineage placement observed in p-Creode trajectories (FIG. 3). The p-Creode analysis was repeated to include rare enteroendocrine cells in the wildtype p-Creode topology and observed that, while enteroendocrine cells segregated with secretory cells, tuft cells still by-and-large shared a trajectory with absorptive cells. To confirm findings with an alternative dataset, a 7,000+-cell scRNA-seq dataset generated using 10× Genomics were re-analyzed, from which the expected distribution of cell types were reproduced. p-Creode analysis demonstrated cell differentiation originated from stem cells, and bifurcated into the absorptive and secretory cells, which further diverged into the Paneth and goblet cell lineages. In addition, observed were non-secretory placement of the tuft cell lineage with the absorptive lineage in 68% of p-Creode topologies and 32% placement with the secretory lineage. Slight discrepancies in the results of the two analyses may be accounted for by technical differences between the datasets in regards to cell isolation, library preparation, or data processing procedures. Furthermore, p-Creode analysis of the TNF^{ΔARE/+} scRNA-seq dataset illustrated that, even under inflammatory conditions, tuft cells can share a trajectory with absorptive cells (FIG. 2F). Quantification of 100 p-Creode runs showed that tuft cell placement was non-secretory in 84% of TNF^{ΔARE/+} maps and secretory in 16% of maps (FIG. 2H). Taken together, these results show that tuft cell lineage branching from a non-secretory route was a robust and consistent feature of the small intestine across multiple datasets. Given that ATOH1 is associated with the secretory cell lineage and the frequency of tuft cell placement outside this lineage, it is implicated that ATOH1-independent tuft cells exist, and are perhaps in the majority, in a microbiome replete intestinal environment.

p-Creode analysis of the antibiotic-treated wildtype dataset were hypothesized to contain only ATOH1-dependent tuft cells, revealed that these tuft cells share a trajectory almost exclusively with secretory cells (FIG. 3). Quantification of 100 p-Creode maps revealed that tuft cells were placed with secretory cells rather than absorptive cells 99% of the time (FIG. 3). Differential gene expression analysis of antibiotic-treated wild type tuft cells revealed increased expression of genes associated with secretory cells, including Mucin (Muc2) and alpha-defensin (Defa22) (SF). In addition, t-SNE analysis showed that these tuft cells belong to the same cluster as enteroendocrine cells, although their memberships do not overlap (FIG. 3C). This similarity to endocrine cells demonstrates shared differentiation of enteroendocrine cells and a tuft cell subset from common Prox1+ progenitors. Finally, all 100 p-Creode graphs generated from AtohKO scRNA-seq data depicted tuft cells and absorptive cells as originating from a common progenitor (FIG. 3), again demonstrating their ATOH1-independence. These ATOH1-independent tuft cells express the canonical tuft cell gene signature, while non-tuft cells in the same condition do not (FIG. 2I), demonstrating that they are not a damage-induced metaplastic cell lineage. Indeed, expression of tuft cell regulators, including Pou2f3, Ptgs1, Ptgs2, Sox4, Sox9, and Trpm5, was confirmed to be expressed in the tuft cell lineage under all conditions (SF). These results further support the existence of ATOH1-dependent (secretory) and ATOH1-independent (non-secretory) tuft cell populations, where the ATOH1-independent population is sensitive to luminal cues and can be induced to expand. Thus, leverage of the AtohKO condition in order to investigate signals that drive ATOH1-independent tuft cell expansion were sought.

Alterations in TCA metabolic pathways within the tuft cell lineage are associated with tuft cell expansion in a microbiome-dependent manner: To identify molecular pathways leading to expansion of ATOH1-independent tuft cells, analysis on dynamic alterations in gene expression along the tuft cell lineage between the wildtype and AtohKO intestinal epithelium were performed, the latter having significant tuft cell expansion. This approach circumvents technical batch effects, since the dynamics of gene expression along a trajectory is self-contained within individual analyses. Dynamics can be illustrated by gradual increases in marker expression along respective intestinal cell lineages.

Genes that switch their expression dynamics between the wildtype and AtohKO conditions were identified. First, different types of gene dynamics in the tuft cell lineage were broadly classified into four categories, as well as a fifth category of unchanged or “flat” dynamics (FIG. 3A). Group one genes, such as Soux, trended upward along pseudo time of the stem-to-tuft cell trajectory, while group four genes, including Rps6, trended downwards. Group one genes included known tuft cell marker genes that are upregulated during differentiation (for instance, Ptgs1 and Sox9), while group four genes included stem cell markers that are downregulated during differentiation. Intermediate genes that trended upwards but returned to a lower baseline or those that trended downwards but returned to a higher baseline were categorized into groups two and three, respectively. When all expressed genes between the wildtype and AtohKO were visualized within these categories, a broad expansion of group two genes was observed upon loss of Atoh1, implicating changes in lineage-specific gene expression dynamics (FIG. 3A). To identify pathways that induced tuft cell specification in an unbiased manner, 1,755 genes that were positively enriched in the AtohKO group were extracted, namely, those that switched categories from a lower category in the wildtype data to a higher category in AtohKO. Over-representation analysis of positively enriched genes in the AtohKO epithelium identified pathways related to the tricarboxylic acid (TCA) cycle and oxidative phosphorylation based on KEGG (FIG. 3B), Wild pathways, and Reactome analyses. This analysis was repeated by grouping the dynamic trends into either “up” or “down” and again identified genes that were positively enriched in the AtohKO trajectory. This coarse grain analysis produced similar results as before, as over-representation analysis identified enrichment for metabolism-associated processes, including the TCA cycle and the electron transport chain (ETC).

The TCA cycle converts glycolysis products into NADH+, which is then shuttled to the electron transport chain for ATP production. Alterations in the wildtype and AtohKO tuft cell lineages of selected pathway genes based on KEGG analysis were directly visualized by plotting expression trends fit to raw data from ten representative p-Creode maps (FIG. 3C-3H). As benchmarks, it was observed that dynamics of tuft and stem cell signature genes were unaltered between wildtype and AtohKO tuft cell trajectories, with Dclk1 and Trpm5 trending upwards, and stem and progenitor genes, Myc and Pcna, trending downwards. In contrast, the TCA cycle enzyme malate dehydrogenase (Mdh2) trended down along the wildtype tuft cell specification trajectory, while its expression remained constant in the AtohKO trajectory (FIG. 3D) Similarly, other TCA enzymes such as Idh3a (FIG. 3C), Sdhb (FIG. 3G), and Sdhd (FIG. 3H), and downstream ETC genes such as those coding NADH dehydrogenases and ATP synthases (FIGS. 3E-3F), all switched to more positive dynamic trends along the AtohKO tuft cell trajectory compared to the wildtype trajectory. Analysis of altered dynamics suggests that tuft cell expansion in the AtohKO model is accompanied by increased TCA cycle and downstream metabolic activities along the tuft cell specification trajectory. As a control, TCA cycle gene expression perturbation in succinate-treated tissues were examined. Relative expression of genes such as Mdh2 and Sdhc was constitutively high in both untreated and succinate-treated enterocytes (SF). In contrast, exogenous succinate treatment induced Idh3b, Idh3a, Sdha, and Sdhd in the tuft cell lineage (SF). Finally, gene expression in the secretory lineage were examined and observed that genes such as Sdhc were significantly reduced upon succinate treatment. These results suggest that succinate treatment induces lineage-specific increase in TCA cycle genes in the tuft cell population.

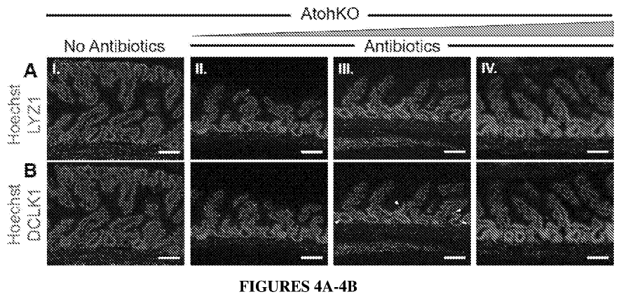

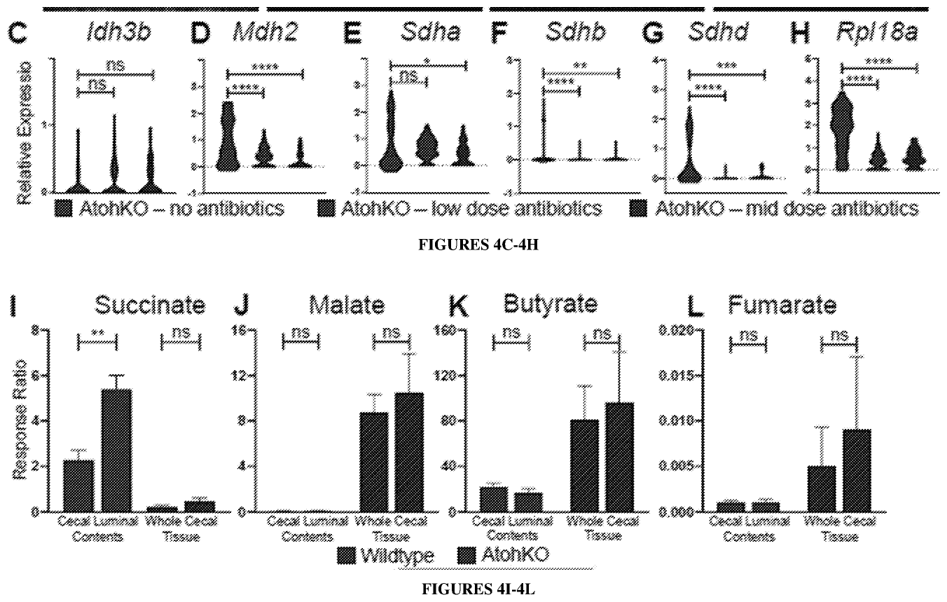

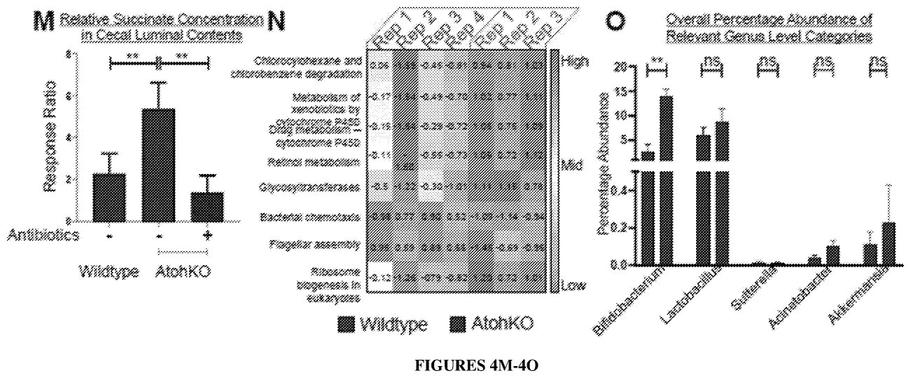

To confirm these results, cells were grouped within the tuft cell trajectory and standard differential expression analysis performed between wildtype and AtohKO cell populations. Standard gene set enrichment analysis (GSEA) performed on genes upregulated in AtohKO tuft cells identified positive enrichment for TCA cycle genes (FIG. 3I). Enrichment plots for the two GSEA gene sets with the highest normalized enrichment score (NES), “Reactome_Citric_Acid_Cycle_TCA_Cycle” and “Mootha_TCA” are shown in FIG. 3 and the expression of highly enriched genes from the former gene set was compared between wildtype and AtohKO tuft cells (FIGS. 3J-3K). TCA cycle-related enzymes Idh3b, Mdh2, Sdha, Sdhb, Sdhd, Sdhc, Citrate synthase (Cs), Idh3a, Idh3g, and Ogdh, as well as the ribosomal protein gene Rpl18a, were all significantly higher in AtohKO tuft cells compared to wildtype tuft cells (FIG. 3N-3S). Additional GSEA over Gene ontology (FIG. 3L), KEGG (FIG. 3M), PANTHER, and Wiki pathways gene sets also identified that TCA cycle-associated genes were upregulated in the AtohKO tuft cells, further suggesting activation of metabolic pathways in this expanded cell population. To support the role of the microbiome in inducing these changes during tuft cell expansion, scRNA-seq data were generated from AtohKO animals on a low to mid dose antibiotic regimen, where ATOH1-independent tuft cells were still being specified, albeit at a suppressed frequency (FIGS. 4BII-4BIII), along with AtohKO tuft cells from vehicle-treated mice, which have significant tuft cell expansion. TCA cycle enzymes Idh3b, Mdh2, Shda, Sdhb, and Sdhd, along with associated Rpl18a, which were all upregulated in vehicle-treated AtohKO tuft cells, were significantly decreased following antibiotic treatment (FIGS. 4C-4H). Idh3a, Idh3g, and Sdhc, which trended upward in the AtohKO tuft cells, trended downward in the antibiotic-treated AtohKO condition, while Cs and Ogdh were increased. Collectively, these results indicate that ATOH1-independent tuft cell expansion is accompanied by increases in TCA cycle gene expression, which is driven by extrinsic signals from the intestinal microbiome to induce tuft cell expansion in vivo.

Non-parasite-derived sources of succinate in the luminal environment drive tuft cell expansion: Given the microbiome dependency and metabolic upregulation observed during ATOH1-independent tuft cell expansion, the differences in (1) metabolites and (2) microbiota composition in the intestines of wildtype and AtohKO animals were characterized. O-benzylhydroxylamine (O-BHA) derivatization was used to analyze metabolite levels in cecal luminal contents and cecal tissue. O-BHA analysis revealed that the relative concentration of succinate was significantly increased in the AtohKO cecal luminal contents but not in cecal tissue, compared to the wildtype condition (FIG. 4I) while levels of malate and butyrate, were not altered in the AtohKO condition (FIGS. 4J, 4K). Succinate or succinic acid is a metabolic intermediate in the TCA cycle and is converted into fumarate by the enzyme succinate dehydrogenase. Fumarate levels were negligible in both the wildtype and AtohKO lumen but trended upwards in the AtohKO tissue, suggestive of host metabolic processing of luminal succinate (FIG. 4L). Moreover, the disparity in succinate concentration between luminal contents and whole tissue were indicative of a commensal microbiota-derived, rather than a host-derived, origin for succinate (FIG. 4I). To support this, the analysis in the AtohKO condition following microbiome depletion was repeated. Succinate levels were significantly decreased in the antibiotic-treated AtohKO cecal luminal contents, confirming that the commensal microbiota was primarily responsible for succinate production in this model (FIG. 4M). It was shown that exogenous succinate administration induces tuft cell expansion, while major basic protein-positive eosinophils and GATA3+cells, components of the type 2 immune response, were increased in both succinate-treated and AtohKO intestinal tissues. Succinate administration did not induce tuft cell expansion in the colon. In contrast to ileal tuft cells, colonic tuft cells are more closely clustered with colonic enteroendocrine cells, similar to in the wildtype antibiotic treated condition (SF). Differences between small intestinal and colonic tuft cells are further highlighted by the absence of succinate receptor (Sucnr1) expression, as well as a significantly lower Il25 expression, components that play a role inducing a type 2 response and subsequent tuft cell expansion. In antibiotic-treated AtohKO animals where ATOH1-independent, microbiome-sensitive tuft cells were not specified due to a suppressed microbiome, succinate administration restored tuft cell expansion to microbiome-replete levels (SF), demonstrating the sufficiency of this commensal-derived metabolite in triggering tuft cells. In contrast, colonic tuft cells, which were suppressed in the AtohKO model with or without antibiotics, were not restored with succinate treatment (SF), consistent with their characterization as ATOH1-dependent tuft cells. These results demonstrate that commensal-derived metabolic signals are necessary and sufficient to induce ATOH1-independent tuft cell expansion in the small intestine.

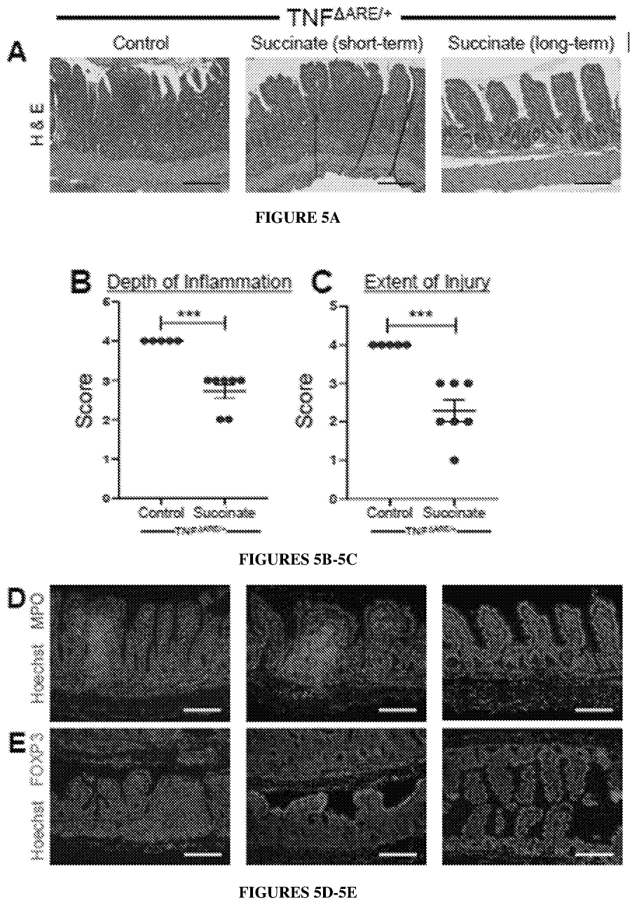

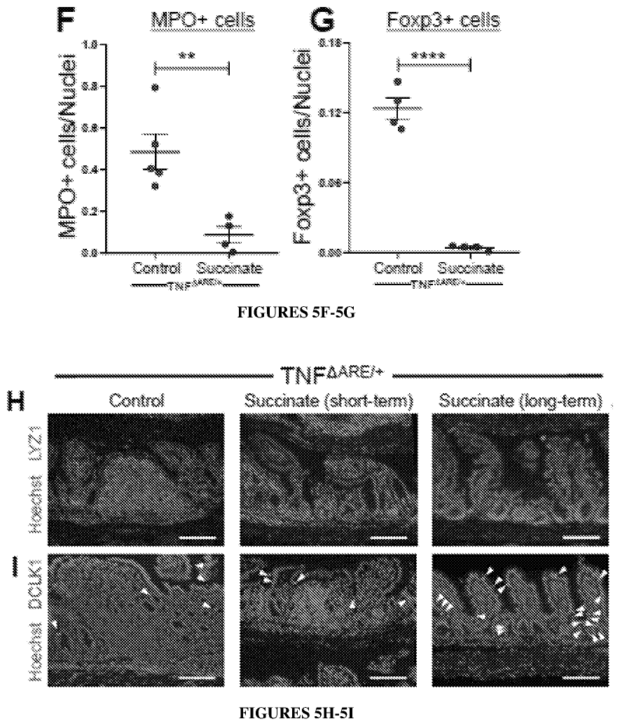

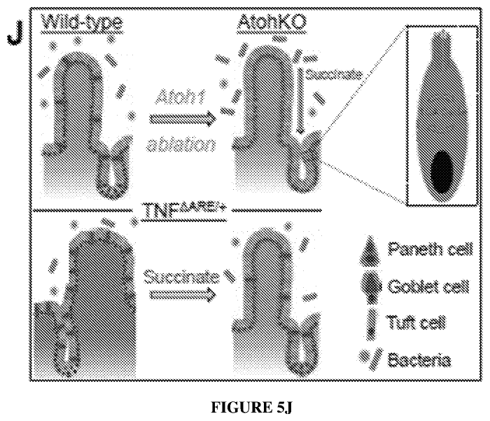

As the microbiome was necessary for in vivo tuft cell expansion, sequencing of the V4 region of the 16S rRNA gene to investigate altered microbiome distribution in the AtohKO intestine was used. DNA extraction and 16S rRNA gene sequencing were performed from the ileal luminal contents of co-housed wildtype and AtohKO littermates. Principal coordinate analysis of beta-diversity measures, including Bray-Curtis, unweighted UniFrac, and weighted UniFrac indices, which measure inter-sample relatedness, demonstrated that wildtype and AtohKO replicates clustered together based on biological phenotype rather than housing. Analysis of microbiome composition revealed a decrease in genus Barnesiella within the AtohKO replicates compared to the wildtype replicates, while the relative abundance of Parasutterella and Bifidobacterium was increased. The latter has been associated with ameliorating symptoms in a variety of gastrointestinal pathologies, including colorectal cancer and IBD. Bifidobacterium infantis, Bifidobacterium breve, and Bifidobacterium pseudolongum are components of the VSL3 probiotic, which has demonstrated the ability to induce remission in a subset of patients with active IBD. PICRUSt inference analysis was performed on 16S gene sequence data and identified eight functional categories that were positively enriched in the AtohKO microbiome, including “Chlorocyclohexane and chlorobenzene degradation” and “Retinol metabolism” (FIG. 4N). A simplified diagram of the Chlorocylohexane and chlorobenzene degradation pathway (ko00361) shows that this process is associated with succinate production. Further analysis revealed that Bifidobacterium, Lactobacillus, Sutterellla, Acinetobacter, and Akkermansia contributed to the positive enrichment of this pathway in the AtohK0 microbiome (FIG. 4O), however only Bifidobacterium was significantly increased compared to the wildtype small intestine (FIG. 4O). Specifically, observed was that Bifidobacterium pseudolongum, a known producer of succinic acid, was increased six-fold in the AtohKO microbiome. To confirm the contribution of the microbiome to tuft cell expansion, germ-free wildtype animals were inoculated with the cecal microbiome from either AtohKO or wildtype animals for a short- (3-day) and long-term (7-day) period, with the caveat that the presence of microbiome-regulating secretory cells will likely normalize the microbiome. DCLK1+ tuft cells were significantly increased in both the duodenum (SF) and the ileum (FIG. 5) after three days of inoculation with AtohKO contents compared to control germ-free animals or those colonized with wildtype contents (SF), although the increase was not as pronounced as other tuft cell expansion conditions. After seven days however, tuft cell numbers were not significantly different between wildtype and AtohKO-colonized animals in either the duodenum (SF) or the ileum (figure), suggesting that the luminal perturbation has been normalized In contrast, colonic tuft cells were not responsive to AtohKO gavage contents after either three or seven days (SF) and were not significantly different from animals that received wildtype contents (SF). To confirm microbial species associated with tuft cell expansion, 16S sequencing of the gavage inoculum were performed and intestinal lumen contents in post-gavage animals. As anticipated, Bifidobacterium pseudolongum trended upward in the AtohKO inoculum compared to the wildtype inoculum (SF). Moreover, B. pseudolongum was enriched at three days post-gavage in the small intestine of AtohKO-gavaged animals in comparison to wildtype-gavaged controls (SF). Abundance of B. pseudolongum was reduced at seven days, suggesting microbiome normalization at that time (SF). Taken together, the findings indicate that the metabolic potential of certain commensal communities can drive ATOH1-independent tuft cell expansion.

Succinate treatment ameliorates inflammation in the TNF^{ΔARE/+} model: Metabolic and microbiome analysis of the AtohKO small intestine indicated that the TCA cycle intermediate succinate was linked to in vivo tuft cell expansion. Given the inverse correlation between tuft cell frequency and inflammation severity in the human and mouse ilea, it was believed that enhanced tuft cell specification could suppress inflammatory disease. Therefore, succinate was therapeutically administered in the drinking water of adult TNF^{ΔARE/+} mice, following disease onset, for a short- (<1 week) and long-term (1 month+) treatment period. While succinate-treated TNF^{ΔARE/+} animals failed to gain as much weight as untreated controls, this can likely be attributed to the palatability of succinate, as wildtype animals treated with succinate also had minimal weight gain. However, succinate treatment in TNF^{ΔARE/+} animals markedly improved intestinal tissue organization compared to age-matched, untreated TNF^{ΔARE/+} controls, based on restored crypt-villus architecture and minimized villus distortion (FIG. 5A). Histological examination demonstrated that succinate-treated animals had decreased mucosal destruction, as assessed by depth of inflammation and extent of tissue injury throughout the length of the ileum (FIG. 5B-C). Additionally, immune cell subsets were assessed, including neutrophils and T-regulatory cells, that are increased in the TNF^{ΔARE/+} model compared to wildtype littermates. MPO+ neutrophils (FIG. 5F) and FOXP3+ T-regulatory cells (FIG. 5G) were significantly reduced in long-term succinate-treated animals, indicative of decreased infiltrative disease. Due to the absence of disease following long-term succinate treatment, immunological assessment on short-term succinate-treated animals in order to capture alterations in immune responses was studied. Multiplex Luminex was performed to assay cytokine levels as succinate treatment did not result in decreased TNF-α levels in the TNF^{ΔARE/+} model (FIG. 6). Short-term succinate treatment induced IL-27 expression, which has been shown to be a potent inhibitor of Type 17 responses during inflammation (citation). Conversely, observed were significantly reduced levels of IL-23, which drives activation of the Th17 lineage from naïve CD4+ cells (citations). A significant increase of two IL-17 isoforms, IL-17A and IL-17F, in short-term succinate-treated animals (FIG. 6) were observed. These animals also had increased levels of IL-22. IL-22 has been shown to enhance mucosal regeneration in inflammation and animals deficient for IL-22 have abrogated worm clearance. Canonically, host response to helminth infection is facilitated by the action of type 2 cytokines which increase mucus production and smooth muscle contraction to drive worm clearance. IL-25 is released by epithelial tuft cells in response to eukaryotic infection while IL-13 and IL-4 are released by innate lymphoid type 2 cells (ILC2s) (citations). Short-term succinate administration in TNF^{ΔARE/+} animals significantly increased levels of IL-25, IL-4, and IL-13, compared to untreated controls (fig). Other cytokines IL-5, IL-9, and IL-10, which activate immune cell recruitment following worm infection were increased (SF). Overall, cytokine assessment implies that succinate-induced reduction of inflammation arises due to decreased activation of type 17 immunity associated with inflammation. These effects were accompanied by an anti-inflammatory profile, analogous to the anti-helminth immune response.

Summary This example presents evidence showing that small intestinal tuft cells possess the capacity to modulate intestinal inflammation. Observed were decreased numbers of tuft cells, labeled with pEGFR and COX2, in ileal tissues acquired from CD patients. These results were confirmed in the TNF^{ΔARE/+} model of Crohn's-like ileitis where frequency of DCLK1+ tuft cells was inversely correlated with inflammation severity. Tuft cell presence was believed to suppress inflammation and, conversely, increasing tuft cell specification may potentially counteract pro-inflammatory signals in the inflamed intestine. To identify mechanisms driving increased specification of tuft cells, a genetically-inducible model of tuft cell expansion, the AtohKO model was developed. scRNA-seq was performed in wildtype, TNF^{ΔARE/+}, and AtohKO animals to examine gene expression changes in small intestinal tuft cells. Analysis of wildtype and TNF^{ΔARE/+} scRNA-seq datasets using the p-Creode algorithm confirmed a non-secretory origin for epithelial tuft cells, independent of the Atoh1-dependent secretory lineage. In the AtohKO model, both trend dynamic and population analysis were applied to demonstrate that expression of TCA cycle genes was upregulated. This shows that tuft cells in the AtohKO model are specified in a more metabolically favorable environment.

Functional analysis of changes in the AtohKO microbiome implicated the production of the TCA cycle intermediate, succinate. Increased levels of Bifidobacterium and succinate were detected in the AtohKO intestine, suggesting a correlation to the tuft cell expansion observed. Several strains of the genus Bifidobacterium are known succinic acid producers and have been linked to establishment of an adult microbiome during infant development. As microbiome depletion in the AtohKO model suppressed tuft cell expansion and decreased expression of TCA cycle genes, microbiome was implicated, based on the enrichment of succinic acid-producing species, to play a role in driving tuft cell specification in the AtohKO model. This example demonstrates that commensal-derived succinate may also induce tuft cell expansion.

To investigate the effect of increased tuft cell specification on inflammation, succinate was therapeutically administered to TNF^{ΔARE/+} animals following onset of disease. Significant histological improvements in succinate-treated TNF^{ΔARE/+} animals were observed, including decreased villus blunting and tissue destruction compared to untreated controls Immune cell infiltration, a hallmark of TNF^{ΔARE/+} ileitis, was significantly reduced based on the absence of MPO+ neutrophils and FOXP3+ T-regulatory cells. Finally, LYZ1+ Paneth cells were restored to the crypts of the ileum, comparable to wildtype controls, and less stromal LYZ1 expression was detected. Furthermore, DCLK1+ tuft cells were increased with succinate treatment in less inflamed TNF^{ΔARE/+} subjects, once more suggesting a role for this cell type in suppressing inflammation. Tuft cells sit at the crossroads of the epithelial, microbiome, and immune systems of the intestinal tract and, therefore, may have the potential to shift the balance between health and disease. These findings demonstrate that enhanced tuft cell specification could be a viable therapeutic strategy for the treatment of inflammatory illnesses.

Butyrate, a short-chain fatty acid (SCFA), produced via bacterial fermentation of dietary fermentation, promotes maturation of colonic T-regulatory cells and is preferentially utilized as an energy source for epithelial colonocytes. A known histone deacetylase inhibitor, butyrate represses growth in proliferative progenitors and stem cells in the base of the colonic crypt. Folate, or vitamin B₉, is essential for human health and is involved in DNA replication and nucleotide synthesis. While mammals cannot produce folate, certain Bifidobacterium and Lactobacillus species can generate this micronutrient from dietary sources. Human subjects colonized with Bifidobacterium and Lactobacillus had increased folate levels as well as altered DNA methylation patterns in intestinal epithelial cells, suggesting that exposure to microbial signals can induce transcriptional changes in the host. Additionally, TCA cycle metabolites have been shown to induce epigenetic changes under favorable metabolic circumstances. For instance, the TCA cycle intermediate α-ketoglutarate is a substrate for a family of demethylase enzymes, while Acetyl-CoA, in nutrient-rich conditions, induces cell growth by directly promoting histone acetylation. Therefore, it is possible that succinate acts in a similar manner to alter transcriptional patterns in the intestinal epithelium and induce downstream immunological changes.

Summary: The use of single-cell RNA-seq to decipher microbial-host crosstalk in intestinal tuft cell specification and compounds and methods of treating IBD are disclosed. In a well-established mouse model (TNFΔARE) of CD, highly inflamed regions with lower tuft cell numbers were observed, while less inflamed regions had more Dclk1+ tuft cells, suggesting an inverse correlation between inflammation and tuft cell specification. A novel, genetically-inducible model of tuft cell hyperplasia (Lrig1^{CreERT2/+}; Atoh1^{fl/fl}—AtohKO) was developed for driving increased tuft cell number in the small intestine. Metabolite analysis was performed and the levels of the citric acid cycle intermediate succinate was observed to be increased in the AtohKO ileum. It was confirmed that antibiotic treatment decreases both tuft cell numbers and levels of succinate, confirming that commensal microbiome and microbial-derived metabolites can drive tuft cell specification in the absence of eukaryotic infections. It was hypothesized that succinate could increase tuft cell numbers and subsequently suppress inflammation in the IBD model. Thus, succinate (120 mM) in the drinking water was administered to TNFΔARE animals therapeutically, beginning treatment after animals had developed disease, and continued for 1 month. Improved histology was observed in succinate-treated animals compared to controls, characterized by less villus blunting and normal villus-crypt architecture. MPO+ neutrophils were decreased following succinate treatment while tuft cells were increased, suggesting decreased disease in these animals Lyz+ Paneth cells, which are decreased in CD and TNFΔARE animals, were increased in Paneth cells. Increasing tuft cell number can be utilized to suppress IBD inflammation and the mechanism by which enhancing tuft cells can ameliorate intestinal inflammation is being developed.

In summary, while anti-inflammatory antibody, anti-steroidal, and microbiome therapies are available for IBD, this work may have greater efficacy with fewer off-target effects.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method for preventing, treating or suppressing Crohn's Disease (CD) in a subject comprising:
administering to the subject a therapeutic effective amount of a pharmaceutical composition that enhance tuft cell specification.

2. The method of claim 1, wherein the pharmaceutical composition comprises succinic acid or a pharmaceutically acceptable salt, ester, solvate, or prodrug thereof.

3. A method for preventing, treating or suppressing an inflammatory bowel disease (IBD) in a subject comprising:
administering to the subject an effective amount of a pharmaceutical composition comprising succinic acid or a pharmaceutically acceptable salt, ester, solvate, or prodrug thereof.

4. The method of claim 3, wherein the inflammatory bowel disease is selected from Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease, or Behcet' s disease.

5. The method of claim 4, wherein the inflammatory bowel disease is Crohn's disease.

6. (canceled)

7. The method of claim 3, wherein the pharmaceutical composition comprises sodium succinate.

8. The method of claim 7, wherein sodium succinate is present in the pharmaceutical composition at a concentration of 100 mM or greater.

9. The method of claim 7, wherein sodium succinate is present in the pharmaceutical composition at a concentration of from 100 mM to 200 mM.

10. The method of claim 3, wherein the pharmaceutical composition is administered orally to the subject.

11. The method of claim 10, wherein the pharmaceutical composition is administered in a food or drink product.

12. The method of claim 3, wherein the pharmaceutical composition is administered daily for at least 5 days to 90 days.

13. The method of claim 3, wherein the pharmaceutical composition is administered in combination with an additional therapeutic agent for treating inflammatory bowel disease.

14. The method of claim 13, wherein the additional therapeutic agent comprises an antibiotic.

15. The method of claim 3, wherein the subject is a human.

16. A method for enhancing tuft cell specification in a subject comprising:
administering to the subject an effective amount of a pharmaceutical composition comprising succinic acid or a pharmaceutically acceptable salt, ester, solvate, or prodrug thereof.

17. The method of claim 16, wherein the tuft cell is intestinal tuft cell.

18. The method of claim 16, wherein the pharmaceutical composition induces expression of IL-25 or IL-13 by the tuft cells.

19. (canceled)

20. The method of claim 16, wherein succinic acid or a pharmaceutically acceptable salt, ester, solvate, or prodrug thereof is delivered orally to the subject.

21. The method of claim 16, wherein the subject has or is predisposed to an inflammatory bowel disease.

22. The method of claim 21, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

23. (canceled)
